(19) United States
(12) Patent Application Publication
(10) Pub. No.: US 2009/0246292 A1
(43) Pub. Date: Oct. 1, 2009

(54) ANTIMICROBIAL FORMULATIONS COMPRISING A QUINONE AND A COPPER SALT

(75) Inventors: Scott Seville, West Yorkshire (GB); Priscila Gottardello, Leeds (GB); Daniel James Fitzgerald, Leeds (GB)

Correspondence Address: Weaver Austin Villeneuve & Sampson LLP, P.O. BOX 70250, OAKLAND, CA, 94612-0250, US

(73) Assignee: SYNTOPIX LIMITED, Bradford (GB)

(21) Appl. No.: 12/279,965

(22) PCT Filed: Feb. 20, 2007
(86) PCT No.: PCT/GB2007/000586

§ 371 Date: Jan. 21, 2009

(30) Foreign Application Priority Data
Feb. 21, 2006 (GB) 0603380.7
Sep. 22, 2006 (GB) 0618691.0

(51) Int. Cl. A61K 33/34; A01N 59/20; A01N 55/02; A61K 31/30; A61K 31/555; A01N 37/18; A61K 38/02; A61P 17/10; A61P 31/00; A01P 1/00

(52) U.S. Cl. 424/632; 424/630; 514/500; 514/184; 424/637; 424/634; 424/635; 514/6

(57) ABSTRACT

An antimicrobial formulation containing a quinone, such as a benzoquinone or hydroquinone, and a copper salt. The formulation may in particular be used against staphylococci or propionibacteria, more particularly to treat skin and skin structure conditions such as acne.

### FIELD OF THE INVENTION

This invention relates to antimicrobial formulations, and to the use of certain combinations of compounds as antimicrobial agents, in particular for the treatment of skin and skin structure conditions such as acne.

### BACKGROUND TO THE INVENTION

Certain quinones, in particular t-butyl hydroquinone (TBHQ), are known for use as antioxidants and in certain contexts as antimicrobial agents.

TBHQ for example has been used as a preservative to stabilise foodstuffs, cosmetics and even adhesives. It has also been recognised as an antimycotic (DE-44 34 312).

Other quinones of various types have been disclosed for use as antimicrobial agents—see JP-2003-267910, JP-09255547 and U.S. Pat. No. 6,228,891- and as preservatives (JP-02202804).

Copper salts have also been used as antimicrobial agents, including in anti-acne formulations—see for example US-2005/0123620 which refers to (although does not exemplify) the use of various polyvalent metal compounds, including copper salts, in the topical treatment of acne and warts; U.S. Pat. No. 6,294,186 which describes a topical antimicrobial composition for the treatment of acne containing a benzoic acid analogue and a metal salt which can be a copper salt such as a halide, sulphate or salicylate; and EP-1 437 124 which describes a topical anti-acne formulation containing a hydroxyacid, a copper salt such as a sulphate or pidolate, a zinc salt, an algae extract and a haloalkynyl carbamate. However, there does not appear to have been any mention of using a copper salt in combination with a quinone as an antimicrobial agent.

It has now surprisingly been found that when quinones such as TBHQ are combined with copper salts, a synergistic effect can be observed on their combined level of antimicrobial activity. As a result, novel antimicrobial formulations can be prepared, in particular for topical application, either with improved efficacy and/or containing lower levels of at least one of the active ingredients than would previously have been thought necessary.

### STATEMENTS OF THE INVENTION

According to a first aspect of the present invention there is provided an antimicrobial formulation containing a quinone and a copper salt.

This formulation is preferably suitable for topical application to, and/or contact with, the skin, in particular human skin. The quinone and the copper salt are therefore preferably contained in a pharmaceutically acceptable vehicle which can safely be applied to, and/or contacted with, the skin and/or other epithelia. Ideally the formulation is suitable for topical application to areas such as the nares, eyes, scalp and/or vagina, and/or to tissue areas within the ears and/or the oral cavity.

A formulation which is “suitable for” topical application may also be adapted for topical application.

Suitable vehicles will be well known to those skilled in the art of preparing topical skin care or pharmaceutical preparations. The vehicle will typically be a fluid, which term includes a cream, paste, gel, lotion, ointment or other viscous or semi-viscous fluid, as well as less viscous fluids such as might be used in sprays (for example for nasal use). The quinone and the copper salt may each independently be present in the form of a solution or suspension, the term “suspension” including emulsions and other multi-phase dispersions.

Either or both of the quinone and the copper salt may, whether separately or together, be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

In the context of the present invention, the term “quinone” means cyclohexadiene-1,4-dione, or any similar compound containing two or more C—O groups in an unsaturated ring. A quinone may be present in the form of a hydroquinone (hydroxyquinone), in which one or more of the C═O groups is instead present as a C—OH group, or as a radical in which one or more of the C═O groups is present as C—O^{•}. It may be present as a mixture of two or more of these forms, for instance as an equilibrium mixture of a benzoquinone and its corresponding hydroquinone.

The two C═O groups or C—OH groups of a (hydro)quinone may be positioned ortho, meta or para to one another, preferably either ortho or para, more preferably para as in p-benzoquinone (cyclohexadiene-1,4-dione) or the corresponding para-substituted hydroquinone HO—Ph-OH.

The quinone may for instance be a benzoquinone (by which is meant an optionally substituted cyclohexadiene dione, typically a cyclohexadiene-1,4-dione or cyclohexadiene-1,2-dione) or its corresponding hydroquinone, by which is meant a compound having an optionally substituted unsaturated 6-membered carbon ring, typically a phenyl ring, substituted with two or more —OH groups. More preferably the quinone is an optionally substituted hydroquinone. As mentioned above, a hydroquinone may be present in the form of a radical in which one or more of the C—OH groups exists as C—O^{•}.

Such compounds may be substituted with one or more other groups such as those selected from alkyl groups (in particular C₁ to C₆ or C₁ to C₄ alkyl groups, for instance methyl, ethyl, isopropyl or t-butyl groups); alkoxy groups (in particular C₁ to C₆ or C₁ to C₄ alkoxy groups such as methoxy or ethoxy); halides such as fluorides, chlorides or bromides; nitro groups —NO₂; and amine groups —NR₂ (where each R is independently either hydrogen or hydrocarbyl), in particular NH₂. The quinone may in particular be an alkyl-substituted hydroquinone or an alkyl-substituted benzoquinone. It may include up to four such substituents, but in particular may be mono- or di-substituted with such groups.

In a formulation according to the invention, the quinone is preferably selected from the group consisting of unsubstituted benzoquinones (in particular p-benzoquinone), unsubstituted hydroquinones (in particular p-hydroquinone), alkyl-substituted benzoquinones and alkyl-substituted hydroquinones. In certain cases it may be preferred for the quinone not to be an unsubstituted hydroquinone.

More preferably the quinone is either an alkyl-substituted benzoquinone or an alkyl-substituted hydroquinone, or a mixture of an alkyl-substituted benzoquinone and its corresponding hydroquinone. Yet more preferably it is an alkyl-substituted hydroquinone.

A hydroquinone may be substituted with one or more alkyl groups. An alkyl group may be either a straight or a branched chain alkyl group. It may be or contain cycloalkyl moieties. It may contain for instance from 1 to 12 carbon atoms, preferably from 1 to 10, more preferably from 1 to 8. Particularly preferred alkyl groups are those selected from C₁ to C₆ alkyl groups, more preferably C₁ to C₅ alkyl groups, yet more preferably C₁ to C₄ alkyl groups, for instance methyl, ethyl, iso-propyl or t-butyl groups.

An alkyl group may be attached to a carbon atom of the cyclohexyl ring or to an oxygen atom (thus replacing the hydrogen atom of a hydroxyl group on the cyclohexyl ring). Preferably it is attached to a carbon atom.

The hydroquinone may be substituted with up to six alkyl groups, more preferably up to four alkyl groups, but in particular may be a mono- or di-alkyl hydroquinone, preferably the former.

The hydroquinone may be substituted with one butyl group, which is preferably present at the 2 position; it may however be substituted with more than one butyl group, for instance two or three or four or even five. A butyl group is preferably a t-butyl group.

The hydroquinone may be substituted with two butyl groups, which preferably occupy the 2 and 5 positions. Again the butyl groups are preferably t-butyl groups.

Instead or in addition, the hydroquinone may be substituted with one hexyl group, which is preferably an O-substituted hexyl group for instance replacing the hydrogen atom of a 1-hydroxy group. The hydroquinone may however be substituted with more than one hexyl group, for instance two or three or even four. A hexyl group is preferably a straight chain hexyl group.

Instead or in addition, the hydroquinone may be substituted with one methyl group, which may for example be present at the 2 or the 5 position; it may however be substituted with more than one methyl group, for instance two or three or four or even five. It may for instance be substituted with three methyl groups, which are preferably present at the 2, 3 and 5 positions.

Instead or in addition, the hydroquinone may be substituted with one propyl group, suitably an iso-propyl group, which is preferably present at the 2 position. The hydroquinone may however be substituted with more than one propyl group, for instance two, three, four or even five. A propyl group is again suitably an iso-propyl group.

Instead or in addition, the hydroquinone may be substituted with one, two, three, four or even five ethyl groups.

Instead or in addition, the hydroquinone may be substituted with one, two, three or even four pentyl (preferably t-amyl) groups.

In particular the hydroquinone may be substituted with three methyl groups and one hexyl group, the hexyl group preferably replacing the hydrogen atom of a 1-hydroxy group and the three methyl groups preferably occupying the 2, 3 and 5 positions.

In particular the hydroquinone may be substituted with one methyl and one iso-propyl group, which preferably occupy the 5 and the 2 positions respectively.

In particular the hydroquinone may be substituted with just one t-butyl group, which is preferably present at the 2 position.

An alkyl-substituted hydroquinone may thus be selected from the group consisting of t-butyl hydroquinone (TBHQ); 1-O-hexyl-2,3,5-trimethyl hydroquinone (HTHQ); 2,5-di-t-butyl hydroquinone; thymohydroquinone (a para-hydroquinone substituted at the 2 position with an iso-propyl group and at the 5 position with a methyl group); and mixtures thereof. It may in particular be t-butyl hydroquinone (TBHQ) which is a para-hydroquinone substituted at the 2 position with a t-butyl group.

In certain cases it may be preferred for the hydroquinone not to be unsubstituted resorcinol. In some cases it may be preferred for the hydroquinone not to be unsubstituted catechol (pyrocatechol).

In some cases it may be preferred for the hydroquinone not to be an alkyl-substituted resorcinol or catechol, in particular an alkyl-substituted resorcinol, more particularly a C₆ to C₉ alkyl-substituted resorcinol, most particularly n-hexylresorcinol.

An alkyl-substituted benzoquinone may be substituted with one or more alkyl groups, an alkyl group being as defined above. Such alkyl groups will be attached to carbon atoms of the cyclohexyl ring.

A benzoquinone may be substituted with up to four alkyl groups, but in particular may be a mono- or di-alkyl benzoquinone, preferably the latter.

Such a benzoquinone is preferably substituted with one methyl group, which is preferably present at the 2 or the 5 position; it may be substituted with more than one methyl group, for instance two or three or even four.

Instead or in addition, the benzoquinone is preferably substituted with one propyl group, which is preferably present at the 2 position; it may be substituted with more than one propyl group, for instance two or three or even four. A propyl group is preferably an iso-propyl group.

In particular the benzoquinone may be substituted with one methyl and one iso-propyl group, which preferably occupy the 5 and 2 positions respectively.

The benzoquinone may be substituted with one butyl group (for instance at the 2 position), or with more than one (for instance two, three or four) butyl groups. A butyl group is preferably a t-butyl group.

The benzoquinone may be substituted with two butyl groups, either or preferably both of which is a t-butyl group. These may for instance occupy the 2 and 5 positions, in particular where the benzoquinone is a para-benzoquinone. They may alternatively occupy the 3 and 5 positions, in particular where the benzoquinone is an ortho-benzoquinone.

Instead or in addition, the benzoquinone may be substituted with one, two, three or even four ethyl groups.

Instead or in addition, the benzoquinone may be substituted with one, two, three or even four pentyl (preferably t-amyl) groups.

Instead or in addition, the benzoquinone may be substituted with one, two, three or even four hexyl groups.

An alkyl-substituted benzoquinone may thus be selected from the group consisting of thymoquinone and its derivatives; 2-t-butyl-p-benzoquinone; 3,5-di-t-butyl-o-benzoquinone; 2,5-di-t-butyl-1,4-benzoquinone; 3-t-butyl-p-benzoquinone; and mixtures thereof. More preferably the benzoquinone is selected from the group consisting of thymoquinone (which is a para-benzoquinone substituted at the 2 position with an iso-propyl group and at the 5 position with a methyl group) and its derivatives such as dithymoquinone; 3,5-di-t-butyl-o-benzoquinone; 2,5-di-t-butyl-1,4-benzoquinone; and mixtures thereof.

In the present context a quinone, in particular a hydroquinone or benzoquinone, and more particularly an alkyl-substituted hydroquinone or benzoquinone, may be present in the form of a dimer, oligomer or polymer, the monomer unit of which is a quinone as defined above.

A quinone used in the formulation of the invention, in particular thymoquinone, dithymoquinone or thymohydroquinone, is ideally used in the form of the isolated quinone (whether naturally or synthetically derived, preferably the latter) rather than as part of a plant extract containing a number of different materials.

Particularly preferred quinones for use in a formulation according to the invention are TBHQ, its benzoquinone equivalent 2-t-butyl-p-benzoquinone (TBBQ) and mixtures thereof.

The quinone may be of the type which is active as an antioxidant.

It may be preferred, in particular when the formulation of the invention is for use against propionibacteria, and/or for the treatment of acne, for the quinone to be a para-benzoquinone or a para-hydroquinone, and/or for it to be mono-substituted, preferably with either alkyl or halogen and more preferably with an alkyl group such as methyl, ethyl, propyl or butyl. It may for example be selected from TBHQ, 2-t-butyl-p-benzoquinone(TBBQ), 2-methyl-p-hydroquinone, 2-methyl-p-benzoquinone, 2-chloro-p-benzoquinone, 2-ethyl-p-hydroquinone and mixtures thereof. It may be selected from TBHQ, TBBQ, 2-methyl-p-benzoquinone, 2-ethyl-p-hydroquinone and mixtures thereof. More preferably it is selected from TBHQ, TBBQ, 2-ethyl-p-hydroquinone and mixtures thereof. Yet more preferably it is selected from TBHQ, TBBQ and mixtures thereof.

It may be preferred, in particular when the formulation of the invention is for use against staphylococci, for the quinone to be a para-benzoquinone or a para-hydroquinone, in particular a para-hydroquinone, and/or for it to be mono-substituted, preferably with an alkyl group such as methyl, ethyl, propyl or butyl. It may for example be selected from TBHQ, TBBQ, 2-methyl-p-hydroquinone, 2-ethyl-p-hydroquinone and mixtures thereof. More preferably it is selected from TBHQ, TBBQ and mixtures thereof.

A formulation according to the invention may contain more than one quinone.

The term “copper salt” includes copper (I), (II) and (III) salts. Preferably the copper salt in a formulation according to the invention is a copper (I) (cuprous) or copper (II) (cupric) salt, more preferably a copper (II) salt. It may be selected for instance from copper carboxylates, copper halides, copper sulphadiazine, copper sulphate (in particular the pentahydrate), copper nitrate, copper carbonate, copper oxide, copper oxychloride, copper hydroxide, copper peptides, copper amino acid salts (eg, copper glutamate, copper aspartate and copper glycinate), copper silicates, copper salts of quinolines—especially hydroxyquinolines—and their derivatives (eg, the copper salt of 8-hydroxyquinoline), copper pyrithione and other copper salts of pyridine thiols, and mixtures thereof.

In one particular embodiment of the invention, the copper salt is a salt of a pyridine thiol, which may for example be a 2-pyridine thiol, 3-pyridine thiol or 4-pyridine thiol, in particular a 2- or 4-pyridine thiol. Such a pyridine thiol may be present in the form of a salt or other derivative, for instance a pyridine thiol oxide or hydroxide. Preferably the copper salt is a salt of a pyrithione (ie, an N-oxide pyridine thiol) or tautomer or derivative thereof. In particular it may be copper-2-pyridinethiol-1-oxide.

A pyrithione may be present in the form of a pyrithione derivative, eg, a molecular and/or ionic complex containing the pyrithione group, such as for example a pyrithione salt or a dimer, oligomer or polymer containing a pyrithione or pyrithione salt monomer (for example, dipyrithione, also known as di-2-pyridinedisulphide-1,1′-dioxide).

Generally speaking the copper salt may be either organic or inorganic.

Suitable copper carboxylates include lactate, citrate, ascorbate, acetate, gluconate, laurate, myristate, palmitate, salicylate, aspirinate, stearate, succinate, tartrate, undecylenate, neodecanoate and ricinoleate.

Suitable halides include copper chloride, copper bromide and copper iodide, preferably the cupric halide (CuHal₂) in each case.

Suitable copper salts may for example be those used in the examples below. Preferred copper salts may be selected from copper sulphate (in particular the pentahydrate), copper aspirinate, copper salicylate, copper pyrithione, copper silicate, the copper salt of 8-hydroxyquinoline, copper gluconate, copper chloride, copper hydroxide and copper acetate, again preferably in the form of the copper (II) salt in each case. More preferred copper salts may be selected from copper sulphate, copper aspirinate, copper salicylate, copper pyrithione, copper silicate, the copper salt of 8-hydroxyquinoline and copper gluconate. Yet more preferred may be copper sulphate and copper silicate.

It may be preferred, in particular when the formulation of the invention is for use against propionibacteria, and/or for the treatment of acne, for the copper salt to be selected from copper sulphate (including in particular the pentahydrate), copper salicylate, copper aspirinate and copper silicate, in particular from copper salicylate, copper aspirinate and copper silicate, more particularly from copper salicylate and copper aspirinate (in each case preferably the copper (II) salt). It may be preferred, in particular when the formulation is for use against propionibacteria, and/or for the treatment of acne, for the copper salt to be selected from copper sulphate (including in particular the pentahydrate) and copper silicate, in each case preferably the copper (II) salt. Of these, the copper sulphate may in some cases be more preferred.

It may be preferred, in particular when the formulation of the invention is for use against staphylococci, for the copper salt to be selected from copper sulphate (including in particular the pentahydrate), copper pyrithione, copper gluconate, copper aspirinate and copper silicate, in particular from copper gluconate, copper aspirinate and copper silicate, more particularly from copper gluconate and copper aspirinate (in each case preferably the copper (II) salt). In such cases it may be preferred for the copper salt not to be either the copper salt of 8-hydroxyquinoline or copper (II) tartrate hydrate.

It may be preferred, in particular when the formulation is for use against staphylococci, for the copper salt to be selected from copper sulphate (including in particular the pentahydrate) and copper silicate, in each case preferably the copper (II) salt. Of these, the copper sulphate may in some cases be more preferred.

The copper salt may be used in an at least partially hydrated form, and may thus be formulated in the presence of an aqueous solvent. Alternatively it may be used in the form of a lipid-soluble salt, suitably in the presence of an organic solvent.

In a formulation according to the invention, both the quinone and the copper salt are present as active (ie, antimicrobially active) agents. Surprisingly, such agents have been found to act together synergistically to inhibit, and often to prevent, microbial activity. In other words, they have been found to increase one another's activity in a manner which can be synergistic compared to the sum of the activities of the two agents individually. This is particularly surprising since certain copper salts, in particular copper (II) salts, are known to act as oxidising agents whereas quinones, in particular hydroquinones, are known for use as anti-oxidants. They might therefore be expected, when combined, to reduce one another's activity.

It is possible that the potentiation of one another's antimicrobial activity by a quinone and a copper salt may be at least partly due to the formation of a reaction product having an antimicrobial activity greater than the sum of those of the individual reactants. The invention may thus embrace an antimicrobial formulation containing a reaction product formed between a quinone and a copper salt, in particular between TBHQ and a copper salt such as copper sulphate, copper pyrithione, copper gluconate, copper salicylate or copper aspirinate; this reaction product may be formed in situ immediately prior to, or at the point of, use.

In a formulation according to the invention the quinone and the copper salt, and their relative proportions, are preferably such as to yield at least an additive level of antimicrobial activity compared to the activities of the individual compounds alone (this is sometimes referred to as an “indifferent” interaction between the compounds). More preferably, the compounds and their relative proportions are such as to yield a synergistic effect on antimicrobial activity, by which is meant that the antimicrobial activity of the combination of the two compounds is greater than the sum of the individual antimicrobial activities of the same amounts of the two compounds used individually. An increased level of activity in these contexts may be manifested by a lower concentration of the compound(s) being needed to inhibit and/or to kill the relevant organism, and/or by a larger zone of inhibition in a disc diffusion assay, and/or by a faster rate of microbial inhibition or killing.

Antimicrobial activity encompasses activity against micro-organisms generally, including bacteria, viruses, fungi, protazoa and algae. It may be inhibitory activity or more preferably biocidal (ie, lethal to the relevant organism). A formulation according to the present invention is preferably active at least as a bactericide, more preferably against bacteria associated with skin or skin-borne infections, yet more preferably against staphylococci and/or propionibacteria and most preferably against strains of Staphylococcus aureus and/or Propionibacterium acnes. In a particularly preferred embodiment of the invention, the formulation is active against bacteria associated with acne, such as P. acnes and in some instances P. granulosum. It may instead or in addition be active against E. coli, listeria, salmonella, pseudomonal bacteria, enterococci, Acinetobacter spp, streptococci in particular group A beta haemolytic streptococci, and/or Candida albicans.

The formulation is preferably active against bacteria, in particular staphylococci and/or propionibacteria, which are wholly or partially resistant to one or more antibiotics, for instance those which are in common clinical use. The formulation is ideally active against MRSA bacterial strains, for example. More particularly the formulation is preferably active against erythromycin-resistant, clindamycin-resistant and/or tetracycline-resistant P. acnes strains of bacteria.

Antimicrobial activity may be measured in conventional manner, for instance using the tests described in the examples below. Generally tests for activity involve treating a culture of the relevant micro-organism with the candidate antimicrobial compound, incubating the treated culture under conditions which would ordinarily support growth of the micro-organism, and assessing the level of growth, if any, which can occur in the presence of the candidate compound.

Preferably the quinone used in the present invention has a minimum inhibitory concentration (MIC), at least against propionibacteria and/or staphylococci, of 200 μg/ml or less, preferably 100 or 50 or even 10 μg/ml or less, such as from 0.5 to 50 μg/ml. Its corresponding minimum biocidal concentration (MBC) is preferably 200 μg/ml or less, preferably 100 or 50 or even 10 μg/ml or less, such as from 0.5 to 50 μg/ml. Suitably the ratio of its MIC to its MBC is from 0.125 to 1, ideally from 0.5 to 1. More preferably the quinone also exhibits such characteristics in the presence of at least one of, preferably two or more of, lipid, salt (sodium chloride) and blood, for instance as tested in the examples below—these are species which can be present at the surface of the skin and hence performance in this context can be indicative of suitability for use in topical skin treatment formulations. Activity in the presence of lipid and sodium chloride can be especially important in the context of acne treatment.

Preferably the copper salt used in the present invention has an MIC, at least against propionibacteria, of 1,000 μg/ml or less, preferably 500 or 250 or 100 or 50 or 20 μg/ml or less, such as from 0.5 to 50 μg/ml. Its corresponding MBC is preferably 1,000 μg/ml or less, preferably 500 or 250 or 100 or 50 μg/ml or less, such as from 0.5 to 50 μg/ml. Suitably the ratio of its MIC to its MBC is from 0.125 to 1, ideally from 0.5 to 1. More preferably the copper salt also exhibits such characteristics in the presence of at least one of, preferably two or more of, lipid, salt (sodium chloride) and serum, for instance as tested in the examples below. Again activity in the presence of lipid and sodium chloride can be especially important for acne treatment formulations.

The concentration of the quinone in the formulation might suitably be 0.1% w/v or greater, preferably 0.5% w/v or greater. Its concentration might be up to 10% w/v, preferably up to 5% w/v, such as from 1 to 5% w/v or from 0.5 to 2% w/v.

The concentration of the copper salt in the formulation might suitably be 0.01% w/v or greater, preferably 0.1% w/v or greater. Its concentration might be up to 10% w/v, preferably up to 2% w/v, such as from 0.1 to 1% w/v.

Due to the presence of the other compound, it may be possible for the concentration of either the quinone or the copper salt, at the site of action when the formulation is applied in vivo, to be less than the MBC, or even than the MIC, of that compound alone. For instance the concentration of at least one of the compounds at this point may be 0.8 or less times its MBC or MIC, such as 0.5 or less, 0.25 or less or 0.125 or less.

Preferably the weight ratio of the quinone in the formulation to that of the copper salt is from 500:1 to 1:500, more preferably from 50:1 to 1:50 or from 50:1 to 1:1, yet more preferably from 20:1 to 1:1, most preferably from 10:1 to 1:1 or from 5:1 to 1:1, such as (in particular when used against propionibacteria) about 2:1.

The formulation of the invention is preferably suitable for, and more preferably adapted for, topical administration to human or animal, in particular human, skin. It may also be suitable for, or adapted for, topical administration to other epithelia such as the nares, scalp, ears, eyes, vagina and oral cavity, in particular the nares and ears. It may take the form of a lotion, cream, ointment, foam, paste or gel or any other physical form known for topical administration, including for instance a formulation which is, or may be, applied to a carrier such as a sponge, swab, brush, tissue, skin patch, dressing or dental fibre to facilitate its topical administration. It may take the form of a nasal spray or of eye or ear drops. It may be intended for pharmaceutical (which includes veterinary) use, for example to treat skin infections or as a prophylactic against infections such as MRSA, and/or for cosmetic or other non-medical care purposes (for example, for general hygiene or cleansing).

The vehicle in which the quinone and the copper salt are contained may be any vehicle or mixture of vehicles which is suitable for topical application; the type chosen will depend on the intended mode and site of application. Many such vehicles are known to those skilled in the art and are readily available commercially. Examples may for instance be found in Williams' “Transdermal and Topical Drug Delivery”, Pharmaceutical Press, 2003, and other similar reference books. See also Date, A. A. et al, Skin Pharmacol. Physiol., 2006, 19(1): 2-16 for a review of topical drug delivery strategies.

As described above, the vehicle may be such as to target a desired site and/or time of delivery of the formulation. It may for instance target the formulation to the skin or hair follicles or to the anterior nares (the latter being particularly suitable when the formulation is used as a preventative treatment against staphylococci or other bacteria), most preferably to the skin. It may delay or otherwise control release of the formulation over a particular time period. Either or both of the quinone and the copper salt may be microencapsulated, for instance in liposomes—particularly suitable liposomes, for topical use, are those made from stratum corneum lipids, eg, ceramides, fatty acids or cholesterol.

In some cases a polar vehicle may be preferred. Where the formulation is intended for use on the skin, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas in particular against staphylococci. It is suitably volatile. In cases the vehicle may be alcohol-based or silicon-based.

By way of example, a lotion or gel formulation may contain a mixture of water, and alcohol such as ethanol or phenoxyethanol and a glycol such as propylene glycol.

The formulation may contain standard excipients and other additives known for use in pharmaceutical or veterinary formulations, in particular topical skin care formulations. Examples include emollients, perfumes, antioxidants, preservatives, stabilisers, gelling agents and surfactants; others may be found in Williams' “Transdermal and Topical Drug Delivery”, supra. For the treatment of acne, however, it may be preferred for the formulation not to contain an emollient.

It may further contain additional active agents such as antimicrobial agents. Where the formulation is intended for topical application to the skin, in particular to treat skin and skin structure infections and/or to treat conditions such as acne or atopic dermatitis, it may additionally contain one or more agents selected from anti-acne agents, keratolytics, comedolytics, anti-inflammatories, anti-proliferatives, antibiotics, anti-androgens, sebostatic agents, anti-pruritics, immunomodulators, agents which promote wound healing and mixtures thereof; it may instead or in addition contain one or more agents selected from sunscreens, moisturisers, emollients and mixtures thereof. Generally speaking a formulation according to the invention may contain one or more agents which enhance the activity of another active agent present in the formulation, or reduce a side effect of such an active, or improve patient compliance on administration of the formulation.

An antimicrobial agent may for example be selected from the group consisting of biocides, disinfectants, antiseptics, antibiotics, antimicrobially active antioxidants and mixtures thereof; it is preferably active as a bactericide, in particular against propionibacteria and/or staphylococci.

It may however be preferred for the quinone and the copper salt to be the only active agents in the formulation, or at least to be the only antimicrobially or antibacterially active agents.

The formulation may be suitable for, more preferably adapted for, use on a surface other than living tissue, for instance to treat floors or walls (whether internal or external), work surfaces or instruments, to disinfect contact lenses or to cleanse hair or teeth or nails so as to reduce microbe levels. It may be suitable for application to growing or harvested crops, foodstuffs, non-living tissue (for instance for use as a preservative) or clothing (for instance for bio-agent decontamination). In these cases the excipients, vehicles and/or other additives included with the quinone and the copper salt may be different to those included in a topical skin care formulation, but again may be conventional as known for use in such contexts.

A formulation according to the invention may be incorporated into, and hence applied in the form of, another product such as a cosmetic, a skin or hair care preparation, a pharmaceutical (which includes veterinary) preparation, a cosmeceutical preparation, a toiletry product (for instance a bath or shower additive or a cleansing preparation), a laundry or other fabric treatment product or an agricultural or horticultural product.

The formulation may be incorporated into another product as a preservative; it may for example be included in a food or beverage, a pharmaceutical preparation, a cosmetic or toiletry product, or a tissue, serum or other body sample, so as to inhibit or prevent microbial activity in the product.

The invention provides, according to a second aspect, a product which incorporates an antimicrobial formulation according to the invention.

The formulation of the invention may be prepared in situ, at or immediately before its point of use, for instance its application to the skin or another surface. Thus according to a third aspect, the present invention provides a kit for preparing an antimicrobial formulation according to the first aspect, the kit comprising a source of a quinone and a source of a copper salt, together with instructions for combining the two compounds so as to make the formulation at or before the point of intended use, and/or for the co-administration of the two compounds to a surface such as the skin. The two compounds may each be present in a suitable respective vehicle.

According to one embodiment, the formulation or kit of the invention may contain both a quinone and a copper salt, each encapsulated (for instance microencapsulated) in a separate delivery vehicle; this might for instance allow their release, and hence their contact with one another, only at the intended site of administration.

A fourth aspect of the invention provides a method for preparing an antimicrobial formulation, which method involves mixing together a quinone and a copper salt, preferably together with a pharmaceutically acceptable vehicle.

According to a fifth aspect of the invention there is provided a formulation (preferably a formulation according to the first aspect of the invention) containing a quinone and a copper salt, for use in the treatment of a skin or skin structure condition which is caused by, transmitted by and/or exacerbated by microbial, in particular bacterial, activity. In the context of the present invention, treatment of a skin or skin structure condition encompasses both therapeutic and prophylactic treatment, of either an infectious or a non-infectious condition, on either human or animal but in particular human skin. It thus preferably involves use of the formulation as a microbiocide, in particular as a bactericide, more particularly against staphylococci and/or propionibacteria and most particularly against propionibacteria.

Skin and skin structure conditions which might be treated according to the fifth aspect of the invention include acne, eczema, superficial infected traumatic lesions, wounds, burns, ulcers, folliculitis, mycoses and other primary and secondary skin and skin structure infections. In particular the formulation may be for use in treating acne or acne lesions (for instance, to reduce acne-related scarring).

Treatment of acne encompasses the treatment and/or prevention of lesions and/or scarring associated with acne. Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment of any of these symptoms.

In general, a formulation according to the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne.

The formulation may also be used as a therapeutic or prophylactic treatment against staphylococci on the skin, which might otherwise cause for example MRSA-associated infections.

In the context of the present invention, “skin condition” may in some cases encompass a condition affecting other epithelial or mucosal surfaces such as in the nares, scalp, vagina, eyes, ears or oral cavity.

Treatment of a condition may involve complete or partial eradication of the condition, removal or amelioration of associated symptoms, arresting subsequent development of the condition, and/or prevention of, or reduction of risk of, subsequent occurrence of the condition.

According to the fifth aspect of the invention, the formulation of quinone and copper salt may be prepared in situ, at or immediately before the point of administration. This aspect of the invention thus pertains to any use of a quinone and a copper salt in the treatment of a microbial skin or skin structure condition, the two compounds being administered either simultaneously or sequentially.

This aspect of the invention may also embrace a formulation containing a quinone and a copper salt, for use in the treatment of any condition which is caused by, transmitted by and/or exacerbated by microbial, in particular bacterial, activity. Such a condition may be for example an oral, ocular, aural, nasal or vaginal condition. Again, treatment of such a condition encompasses both therapeutic and prophylactic treatment, of either an infectious or a non-infectious condition, in either a human or an animal but in particular a human. In particular it encompasses the prophylactic treatment of any area of the body, in particular the skin or nares or another epithelial or mucosal surface, against micro-organism infections, including against staphylococcal infections such as those associated with MRSA.

According to a sixth aspect, the invention provides the use of a quinone and a copper salt in the manufacture of a medicament for the treatment of a condition, in particular a skin or skin structure condition, which is caused by, transmitted by and/or exacerbated by microbial (especially bacterial) activity, in particular acne.

The invention further provides, according to a seventh aspect, the use together of a quinone and a copper salt, as an antimicrobial agent, in particular as a bactericide, or in the manufacture of an antimicrobial or specifically antibacterial formulation.

An eighth aspect provides a method for controlling the growth of a micro-organism, in particular a bacterium, the method comprising applying, to an area infected or suspected to be infected or capable of becoming infected with the micro-organism, a combination of a quinone and a copper salt. Again the two compounds may be applied simultaneously or sequentially.

“Controlling the growth” of a micro-organism embraces inhibiting or preventing its growth, whether completely or partially, as well as killing either completely or partially a culture of the organism. It also embraces reducing the risk of subsequent growth of the organism in the area being treated. The method of the invention may thus be used to treat an existing occurrence of the organism or to prevent a potential subsequent occurrence.

Again the area to which the quinone and the copper salt are applied will typically be a surface such as human or animal tissue, in particular the skin or nares, typically of a living human or animal. In this case the quinone and the copper salt may be applied for therapeutic purposes or for non-therapeutic (eg, purely cosmetic) purposes. Thus the method of the eighth aspect of the invention encompasses a method of treatment of a human or animal patient suffering from or at risk of suffering from a condition which is caused by, transmitted by and/or exacerbated by microbial, in particular bacterial, activity, especially a skin or skin structure condition such as acne, the method involving administering to the patient a therapeutically (which term includes prophylactically) effective amount of an antimicrobial formulation containing a quinone and a copper salt.

Alternatively the quinone and the copper salt may be applied to a non-living surface such as in a hospital or food preparation area, a surgical or other instrument or an item of clothing or bedding. For example the method of the eighth aspect of the invention may be used to treat work surfaces, surgical instruments, surgical implants or prostheses, contact lenses, foods, crops, industrial plant, floors (both internal and external), clothing and many other surfaces.

The method of the eighth aspect of the invention preferably involves applying a formulation according to the first aspect.

According to a ninth aspect, the invention provides a method for controlling the growth of a micro-organism, in particular a bacterium, in a product which contains or is suspected to contain or is capable of containing the micro-organism, the method comprising incorporating into the product a combination of a quinone and a copper salt. The product may for example be a food or beverage, a pharmaceutical (which includes veterinary) preparation, a cosmetic or toiletry product, or an agricultural or horticultural product.

Thus the method of the ninth aspect of the invention may be used to preserve all manner of products, or it may be used in the sanitation of food or water supplies or the disinfection of farming areas.

A tenth aspect of the invention provides the use of a quinone in an antimicrobial formulation, in combination with a copper salt, for the purpose of increasing the antimicrobial (in particular antibacterial) activity of the formulation and/or of reducing the amount of the copper salt in the formulation without undue loss of antimicrobial activity.

An increase in antimicrobial activity may be as compared to that of the copper salt alone, at the same concentration as used when combined with the quinone. Ideally the increase is as compared to the sum of the activities of the quinone and the copper salt individually, again at the same respective concentrations as used when the two are combined.

A reduction in the amount of the copper salt in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the copper salt. According to the invention, the quinone may therefore be used for the dual purposes of reducing an undesired property of a formulation containing a copper salt, without undue loss of antimicrobial activity.

Preferably the quinone is used without any reduction in antimicrobial activity compared to the level exhibited by the formulation prior to addition of the quinone. More preferably it is used to give an increase in antimicrobial activity. It may however be used to reduce the amount of the copper salt present, and/or its associated side effects, whilst maintaining the antimicrobial activity of the resultant formulation at a level, albeit lower than that which it would otherwise have exhibited, which is still acceptable in the context of its intended use.

An eleventh aspect of the invention provides the use of a copper salt in an antimicrobial formulation, in combination with a quinone, for the purpose of increasing the antimicrobial (in particular antibacterial) activity of the formulation and/or of reducing the amount of the quinone in the formulation without undue loss of antimicrobial activity.

An increase in antimicrobial activity may be as compared to that of the quinone alone, at the same concentration as used when combined with the copper salt. Ideally the increase is as compared to the sum of the activities of the copper salt and quinone individually, again at the same respective concentrations as used when the two are combined.

A reduction in the amount of the quinone in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the quinone. According to the invention, the copper salt may therefore be used for the dual purposes of reducing an undesired property of a formulation containing a quinone, without undue loss of antimicrobial activity.

Preferably the copper salt is used without any reduction in antimicrobial activity compared to the level exhibited by the formulation prior to addition of the copper salt. More preferably it is used to give an increase in antimicrobial activity. It may however be used to reduce the amount of the quinone present, and/or its associated side effects, whilst maintaining the antimicrobial activity of the resultant formulation at a level, albeit lower than that which it would otherwise have exhibited, which is still acceptable in the context of its intended use.

One copper salt which has been found to be of particular use as an antimicrobial agent is the copper salt of 8-hydroxyquinoline. Accordingly, a twelfth aspect of the present invention provides the copper salt of 8-hydroxyquinoline (“copper 8-HQ”), or a (preferably pharmaceutically acceptable) derivative thereof, for use as an antimicrobial agent. In particular the copper 8-HQ or derivative may be for use as an antibacterial agent, more particularly against propionibacteria and/or staphylococci, especially propionibacteria. It may be for use in the treatment of a condition affecting the human or animal body, which condition is caused by, transmitted by and/or exacerbated by microbial, in particular bacterial, activity. The condition may for example be a skin or skin structure condition, in particular acne. Thus the copper 8-HQ or derivative may be for use in a therapeutic or prophylactic treatment against one or more bacteria associated with acne, or against staphylococci on the skin, nares or another epithelial or mucosal surface.

The copper 8-HQ or derivative may be for use against one or more strains of Staphylococcus aureus and/or Propionibacterium acnes. In a particularly preferred embodiment of the invention, it is for use against bacteria associated with acne, such as P. acnes and in some instances P. granulosum.

It may be for use against one or more bacteria, in particular staphylococci and/or propionibacteria, which are wholly or partially resistant to one or more antibiotics, for instance those which are in common clinical use. It may for example be for use against MRSA bacterial strains. More particularly it may be for use against macrolide-lincosamide-streptogramin (MLS) resistant and/or macrolide-lincosamide-streptogramin-ketolide (MLSK) resistant strains of bacteria, for example propionibacteria.

A quinoline is an aromatic nitrogen-containing compound with a double ring structure containing a benzene ring and a pyridine ring fused at two adjacent carbon atoms. Derivatives of quinoline are used as drugs (especially anti-malarials), fungicides, biocides, dyes and flavouring agents. They are also used as catalysts, corrosion inhibitors and preservatives and as solvents for resins and terpenes.

Various hydroxyquinolines are known in certain contexts to have antimicrobial activity. For example, 8-hydroxyquinoline (also known as oxyquinoline and 8-quinolinol) has been used as an antiseptic agent, while the 8-hydroxyquinoline derivatives 5-chloro-8-hydroxyquinoline (USAN designation: Cloxyquin), 5-chloro-7-iodo-8-hydroxyquinoline (Clioquinol) and 5,7-dichloro-2-methyl-8-hydroxyquinoline (Chlorquinaldol) are available as topical antibacterial and/or antifungal agents. Copper 8-hydroxyquinoline (Cu-8-HQ) (CAS No. 10380-28-6) is a fungicide used in textiles, pain and paper and in agriculture.

Although copper salts have also been included in antimicrobial and anti-acne formulations, as described above, there does not appear to have been any mention of using a copper quinoline salt in this context.

It has now surprisingly been found, however, that the copper salt of 8-hydroxyquinoline is extremely active as an antimicrobial agent, in particular against a wide range of strains of both propionibacteria (the bacteria implicated in acne) and staphylococci. This was not necessarily predictable since many other copper salts have been found, on their own, to be relatively inactive against staphylococci, and/or to lack the wide ranging activity which we have now demonstrated for copper 8-HQ.

A thirteenth aspect of the invention provides the use of copper 8-HQ, or a pharmaceutically acceptable derivative thereof, as an antimicrobial agent in the manufacture of a medicament for the treatment of a condition which is caused by, transmitted by and/or exacerbated by microbial, in particular bacterial, activity. Again the medicament may be for the treatment of a skin or skin structure condition such as acne. It may be for the treatment of a staphylococcal infection.

“Copper 8-HQ”, as used herein, means the copper salt of 8-hydroxyquinoline, which comprises a copper (II) ion complexed to two hydroxyquinoline moieties. The compound is also known as copper 8-quinolinol; copper 8-hydroxyquinolinate; copper 8-quinolate; copper 8-quinolinol; copper 8-quinolinolate; copper bis(8-hydroxyquinolinate); copper oxinate; copper oxine and copper oxyquinolate, among other synonyms.

Derivatives of copper 8-HQ include pharmaceutically acceptable derivatives such as salts, and also so-called “prodrug” forms which revert to an active form of the compound at an appropriate time on or after administration.

In accordance with the invention, the copper 8-HQ or derivative is used as an active agent (ie, antimicrobially active and/or active against acne (which includes against a symptom and/or a cause of acne) and/or active against one or more micro-organisms associated with acne). It may be used as the sole antimicrobially active, or at least the sole antibacterially active and/or the sole anti-acne active agent in a formulation for the treatment of a microbial condition such as acne. In cases it may be preferred for the copper 8-HQ to be used in the absence of a quinone, in particular a benzoquinone or hydroquinone such as an alkyl-substituted benzo/hydroquinone and more particularly t-butyl hydroquinone (TBHQ).

A fourteenth aspect of the present invention provides a method for controlling the growth of a micro-organism, in particular a bacterium such as a propionibacterium or staphylococcal bacterium, the method comprising topically applying, to a surface which is infected or suspected to be infected or capable of becoming infected with the micro-organism, copper 8-HQ or a (preferably pharmaceutically acceptable) derivative thereof.

A fifteenth aspect of the invention provides an antimicrobial formulation which is suitable and/or intended and/or adapted for (preferably adapted for) topical administration, in particular to the skin, the formulation including copper 8-HQ or a derivative thereof. Suitably the copper 8-HQ or derivative is the only antimicrobially active ingredient in the formulation.

Preferred features of the twelfth to the fifteenth aspects of the invention—for example regarding the way in which, concentration at which and/or ingredients with which the copper 8-HQ is formulated, and/or its mode or context of use, and/or the nature of its activity—may be as described above in connection with the preceding aspects. In particular, the copper 8-HQ is suitably administered topically to the skin of a patient. It may be formulated in particular as a suspension or other form of dispersion, for instance in the form of a gel or cream.

Preferred features of the second and subsequent aspects of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Moreover unless stated otherwise, any feature disclosed herein may be replaced by an alternative feature serving the same or a similar purpose.

The present invention will now be further described with reference to the following non-limiting examples and the accompanying illustrative drawings.

### BRIEF DESCRIPTION OF THE DRAWINGS

FIG. 1 is an isobologram showing FIC (fractional inhibitory concentration) values for mixtures of TBHQ and copper sulphate against a propionibacterial strain, as referred to in Example 2 below; and

FIG. 2 is an isobologram showing FIC values for mixtures of TBHQ and copper sulphate against a staphylococcal strain, as referred to in Example 4.

### DETAILED DESCRIPTION

Experimental tests were conducted to determine the antimicrobial activity of formulations according to the invention. As a comparison, the antimicrobial activities of formulations containing a quinone or a copper salt alone were also measured.

Test Micro-Organisms

Several types of test micro-organism were used, representing the staphylococci and propionibacteria genera.

The principal propionibacterial strain used was Propionibacterium acnes NCTC 737. This is the type strain of the genus; it is fully susceptible to antibiotics.

The propionibacteria are clinically significant due to their involvement in acne. This is a very common, complex and multi-factorial skin disease in which P. acnes and other Propionibacterium spp. (for example P. granulosum) play key roles. They are also opportunistic pathogens in compromised hosts. Thus, activity observed against these micro-organisms is expected to be a good predictor of activity against acne.

The principal staphylococcal strain used was Staphylococcus aureus ATCC 29213. S. aureus and other staphylococci are common causes of a wide range of skin, skin structure and wound infections; S. aureus is also known to exacerbate eczema. The ATCC 29213 strain is known to be susceptible to beta-lactam antibiotics such as methicillin.

Activity observed against these micro-organisms is expected to be a reasonable qualitative predictor of antimicrobial activity generally, in particular of activity against micro-organisms responsible for skin and skin structure infections.

Other propionibacterial and staphylococcal strains were also tested, as described in Examples 7, 12 and 13 below. These included certain antibiotic resistant propionibacteria, such as the two P. acnes strains designated PRP-010 and PRP-039 which are resistant respectively to macrolides-lincosamides-streptogramins-ketolides (MLSK) and to macrolides-lincosamides-streptogramins (MLS) and tetracycline—in other words, PRP-010 is resistant to erythromycin and clindamycin, and PRP-039 to erythromycin, clindamycin and tetracycline. They also included certain antibiotic resistant staphylococci, such as the methicillin resistant S. aureus (MRSA) strains EMRSA-15 and EMRSA-16, both available from the Central Public Health Laboratory (CPHL), Colindale, UK. These strains are resistant not only to all beta-lactams but also to a number of other antibiotics in clinical use, making them a serious threat to human health. They are also responsible for the majority (>95%) of hospital-acquired MRSA infections in the UK.

In addition, certain strains of P. granulosum, another bacterium involved in acne, were also tested in Examples 7 and 12. Other staphylococcal species, namely S. simulans, S. xylosus, S. cohnii, S. haemolyticus, S. warneri, S. capitis, S. hominis, S. auricularis, S. saprophyticus and S. epidermidis were also tested in Example 13.

The propionibacteria were cultured and maintained on Wilkins-Chalgren Anaerobe Medium (agar and broth) at pH 6.0; all cultures were incubated anaerobically at 37° C. for 72 hours.

The staphylococci were cultured and maintained on Mueller-Hinton Medium (agar and broth) at pH 7.2; cultures were incubated aerobically at 37° C. for 19-20 hours.

The following tests were carried out to assess antimicrobial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (e.g., 1000, 500, 250, 125 . . . μg/ml, etc. down to 0.49 μg/ml). The culture media were as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, ie, the lowest concentration for which the liquid in the well remained clear.

The assays included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Bactericidal Concentration (MBC) Assay

This assay, normally carried out after an MIC assay, determines the minimum concentration of a compound that is lethal to the micro-organism being tested. Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for bacterial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Agar Dilution MIC Assay

This is a standard international method for quantitatively assessing the antimicrobial activity of a compound in a solid medium. The test compound was prepared to 40× the highest concentration required (e.g., 10 mg/ml for a final concentration of 250 μg/ml) and a series of doubling dilutions were performed in a suitable solvent. A set amount of these antimicrobial stock solutions was then added to molten agar medium (ca. 55° C.), mixed thoroughly, poured into sterile Petri dishes and allowed to cool/set. The culture medium was as described above.

A Multipoint™ Inoculator (AQS Manufacturing Ltd, UK) was used to inoculate the plates by spotting the inocula onto the surface of the agar, delivering approximately 1 to 2 μl per spot (yielding 10⁵ CFU (colony forming units) per spot).

The plate(s) were then incubated under the conditions described above, following which they were examined visually for signs of bacterial growth. The MIC value was ascertained when there was a marked reduction in, or total loss of, growth on the test plate at the lowest concentration as compared to that of the growth on the control plate.

The assays included a positive control (culture medium, diluting solvent and inoculum).

(d) Disc Diffusion Assay (DDA)

This is an internationally recognised standard method for qualitatively assessing the antimicrobial activity of a compound.

A sterile paper disc was impregnated with a sample of the test compound in a suitable solvent and 30 minutes allowed for the solvents to evaporate (where possible). The disc was then placed on an agar plate onto which the test micro-organism had been inoculated. The plate was then incubated under the conditions described above, following which it was examined visually for signs of bacterial growth. If the test compound had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

(e) Synergy Disc Diffusion Assay (SDDA)

This is a variation on the DDA method, in which two compounds are tested together for their combined antimicrobial activity.

Two test compounds A and B were placed on a single paper disc and the above described DDA procedure repeated. An increase in diameter of the zone of inhibition, compared to the greater of the zone diameters for the two compounds individually, was taken to indicate potential antimicrobial synergy. In practical terms, an increase of greater than 5 mm could be treated as significant.

(f) Supplemented Disc Diffusion Assays

Either the DDA or the (S)DDA tests may be carried out using an agar gel supplemented with lipid, salt and/or serum to simulate some of the major components present in human skin and to assess whether these substances might reduce the antimicrobial activity observed for the test compounds. Performance under these conditions can provide a more reliable indication of activity on topical application. For assays using Propionibacterium spp. strains the supplements used in the examples below were lipid (either Tween™ 80 or triolein, both at 1% v/v) and sodium chloride (100 mM). For assays using S. aureus ATCC 29213 the supplements used were sodium chloride (100 mM), lipid (Tween™ 80 at 1% v/v) and horse serum (5% v/v).

(g) Fractional Inhibitory Concentration (FIC) Assay

This assay was used to determine the mode of interaction between two antimicrobial compounds A and B. It was similar to the MIC assay, utilising a 96-well microtitre plate and liquid culture medium. The test compounds were added together to each well at a range of concentrations starting at their respective MIC values and descending in doubling dilutions as with the MIC assay. Typically an 8×8 array of wells could be used to combine 8 different concentrations of compound A (from its MIC downwards, including zero) with 8 different concentrations of compound B (ditto).

The wells were inoculated with freshly grown micro-organism and incubated under the conditions described above.

As for the MIC assay, the results were read by the naked eye. A minimum inhibitory concentration was recorded for each combination of A and B. A fractional FIC index (FICI) was then calculated for each compound in that mixture, and these two indices were added together to give an overall FICI indicative of the mode of interaction.

Thus for each mixture tested, the FIC for compound A (FIC_{A})=MIC for (A+B)/MIC for A alone. Similarly the FIC for compound B (FIC_{B})=MIC for (A+B)/MIC for B alone. The overall FICI═FIC_{A}+FIC_{B}.

An FICI of 0.5 or less was taken to indicate synergy, a value from 0.5 to 4.0 an indifferent effect and values greater than 4.0 antagonism (ie, the two compounds counter one another's activity, leading overall to a diminished antimicrobial effect) (see Odds FC, “Synergy, antagonism, and what the chequerboard puts between them”, J Antimicrob Chemother, 2003; 52:1). These results can be depicted visually on a plot (isobologram) of FIC_{A} against FIC_{B} for the mixtures tested.

### Example 1

### Activity Against Propionibacterium spp (Disc Diffusion Assays)

The following experiments all used P. acnes NCTC 737 as the test organism.

MIC, MBC and DDA assays, as described above, were carried out using the test compounds t-butylhydroquinone (TBHQ, an alkyl-substituted hydroquinone) and aqueous copper (II) sulphate (both sourced from Sigma-Aldrich).

The two compounds were then subjected in combination to the SDDA assay described above, including in the presence of salt and lipid (SL SDDA measurement). Increases in zone diameter (mm) were measured with respect to those observed for the copper sulphate (ie, the compound showing the larger zone diameters during the previous disc diffusion assays on the individual compounds).

For all the (S)DDA experiments, 200 μg of each test compound was loaded onto each disc. The TBHQ was dissolved in ethanol and the copper sulphate in deionised water.

MIC/MBC experiments were performed in duplicate and (S)DDA experiments in triplicate.

The results of the tests on both TBHQ and copper sulphate alone, together with those for the combination, are shown in Table 1 below.

TABLE 1

| | Copper (II) | | Copper sulphate + |
|---|---|---|---|
| Assay | sulphate | TBHQ | TBHQ |
| MIC (μg/ml) | 15.6 | 7.8 | — |
| MBC (μg/ml) | 31.25 | 7.8 | — |
| MIC/MBC ratio | 0.5 | 1 | — |
| (S)DDA (mm) | 51.73 (±1.48) | 12.20 (±1.13) | 63.41 (±0.48) |
| SL (S)DDA (mm) | 51.10 (±3.17) | 0.0 | 62.89 (±1.36) |
| SDDA increase | — | — | 11.68 |
| (mm) | | | |
| SL SDDA increase | — | — | 11.79 |
| (mm) | | | |
| SDDA area increase | — | — | 50.26 |
| (%) | | | |
| SL SDDA area | — | — | 51.44 |
| increase (%) | | | |

(SL (S)DDA = (S)DDA with salt and lipid (1% (v/v) Tween 80) supplements

These data show that both TBHQ and copper sulphate alone are active against P. acnes. When the quinone and the copper salt are combined, however, the data demonstrate a synergistic antimicrobial interaction between the two, with a significant increase in zone diameter over that exhibited by either compound alone. This synergy is maintained under the supplemented conditions.

### Example 2

### Activity Against Propionibacterium spp (FIC Assays)

Mixtures of TBHQ and aqueous copper (II) sulphate, containing various relative proportions of the two actives, were then subjected to FIC assays against P. acnes NCTC 737, as described above. The results were used to prepare FIC isobolograms. All assays were conducted in triplicate.

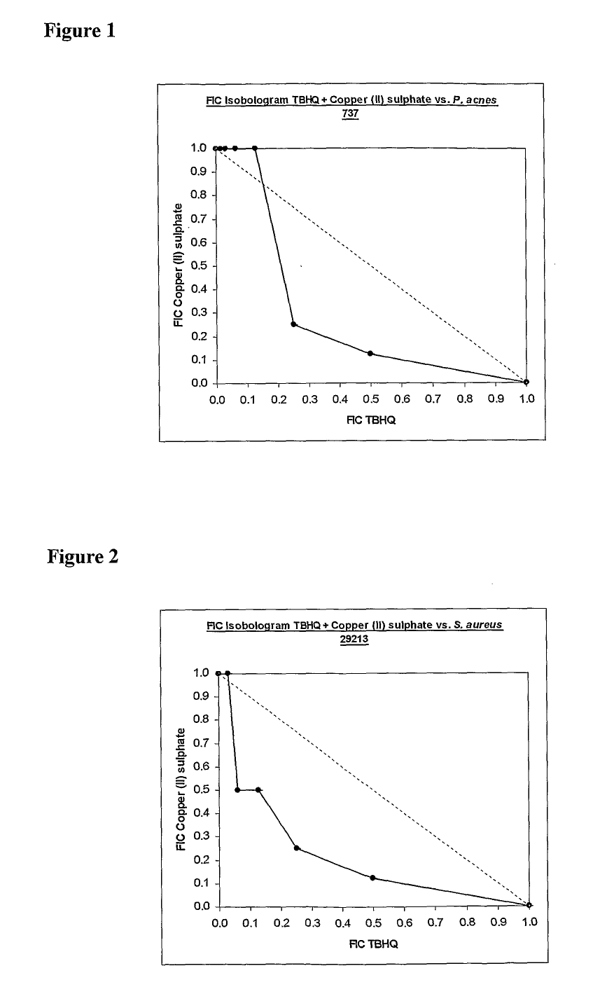

The overall FICI obtained for the mixtures was 0.5, representing the mean of three replicates. A representative isobologram is shown in FIG. 1; the dashed line indicates where overall FICIs (ie, FIC_{TBHQ}+FIC_{copper (II) sulphate}) equal 1, which would indicate a purely indifferent interaction. FIG. 1 clearly demonstrates the synergistic activity of the combination of the quinone and the copper salt against P. acnes NCTC 737.

### Example 3

### Activity Against S. aureus (Disc Diffusion Assays)

The following experiments all used S. aureus ATCC 29213 as the test organism.

MIC, MBC and DDA assays, as described above, were carried out using the test compounds TBHQ and aqueous copper (II) sulphate, as in Example 1.

The two compounds were then subjected in combination to the SDDA assay described above, including in the presence of salt and serum (SS SDDA measurement). Increases in zone diameter (mm) were measured with respect to those observed for the TBHQ (the compound showing the larger zone diameters during the previous disc diffusion assays on the individual compounds).

For all the (S)DDA experiments, 200 μg of each test compound was loaded onto each disc. The TBHQ was dissolved in ethanol and the copper sulphate in deionised water.

MIC/MBC experiments were performed in duplicate and (S)DDA experiments in triplicate.

The results of the tests on both TBHQ and copper sulphate alone, together with those for the combination, are shown in Table 2 below.

TABLE 2

| | Copper (II) | | Copper sulphate + |
|---|---|---|---|
| Assay | sulphate | TBHQ | TBHQ |
| MIC (μml) | 1000 | 3.9 | — |
| MBC (μg/ml) | 1000 | 7.8 | — |
| MIC/MBC ratio | 1 | 0.5 | — |
| (S)DDA (mm) | 0 | 40.42 (±0.89) | 55.37 (±0.79) |
| SS (S)DDA (mm) | 0 | 43.68 (±1.02) | 47.16 (±0.79) |
| SDDA increase (mm) | — | — | 14.95 |
| SS SDDA increase | — | — | 3.48 |
| (mm) | | | |
| SDDA area increase | — | — | 87.65 |
| (%) | | | |
| SS SDDA area | — | — | 16.57 |
| increase (%) | | | |

(SS (S)DDA = (S)DDA with salt and serum supplements)

These data show that TBHQ alone is active against S. aureus. Copper sulphate alone however does not appear to be as active as the quinone, and appears to be inactive against the organism in the disc diffision assays, at the concentration tested.

Surprisingly, when the quinone and the copper salt are combined, the data demonstrate a synergistic antimicrobial interaction between the two, with a significant increase in zone diameter over that exhibited by either compound alone.

### Example 4

### Activity Against S. aureus (FIC Assays)

Mixtures of TBHQ and aqueous copper (II) sulphate, containing various relative proportions of the two actives, were then subjected to FIC assays against S. aureus ATCC 29213, as described above. The results were used to prepare FIC isobolograms. All assays were conducted in triplicate.

The overall FICI obtained for the mixtures was 0.5, representing the mean of three replicates. A representative isobologram is shown in FIG. 2; the dashed line indicates where overall FIC indices (ie, FIC_{TBHQ}+FIC_{copper (II) sulphate}) equal 1, which would indicate a purely additive effect. FIG. 2 clearly demonstrates the synergistic activity of the combination of the quinone and the copper salt against S. aureus ATCC 29213.

### Example 5

### Activity Against P. acnes (Other Copper Salts)

A series of copper salts was tested with TBHQ against P. acnes NCTC 737 using (S)DDA tests as described in Example 1. The MIC and MBC values for each copper salt were also measured as in Example 1 along with (S)DDAs conducted in the presence of salt and/or lipid.

For the (S)DDA experiments, 200 μg of each test compound was loaded onto each disc, with the exception of copper (II) silicate which was added at 62 μg/disc. The copper salts were dissolved in ethanol with the following exceptions: the 8-hydroxyquinoline copper (II) salt, copper (I) iodide, copper (II) pyrithione, copper (II) salicylate and copper (II) aspirinate were dissolved in DMSO; the copper (I) acetate, copper (II) acetate, copper (II)-D gluconate and copper (II) sulphate pentahydrate were dissolved in deionised water; the copper (II) tartrate hydrate was dissolved in 1 M sodium hydroxide; and the copper (II) silicate was used in the form of a 0.62% (w/w) solution in water.

All compounds were sourced from Sigma-Aldrich with the exception of copper (II) silicate (Convé plc, UK), copper (II) pyrithione (3B Medical Systems, Inc., USA), and copper (II) salicylate and copper (II) aspirinate which were produced in-house.

MIC and MBC experiments were conducted in duplicate, supplemented DDA experiments as single replicates and (S)DDA experiments in triplicate.

The MIC, MBC and DDA results are shown in Tables 3 and 4 below and the SDDA results in Tables 5 (unsupplemented assays) and 6 (supplemented assays). All results are collated from a number of experiments.

TABLE 3

| | MIC | MBC | MIC/MBC |
|---|---|---|---|
| Copper salt | (μg/ml) | (μg/ml) | ratio |
| 8-hydroxyquinoline copper (II) salt | 3.9 | 15.6 | 0.25 |
| Copper (I) bromide | 15.6 | 125 | 0.125 |
| Copper (I) iodide | 7.8 | 31.25 | 0.25 |
| Copper (I) oxide | 1.95 | 31.25 | 0.06 |
| Copper (II) acetate | 7.8 | 31.25 | 0.25 |
| Copper (II) tartrate hydrate | 15.6 | 62.5 | 0.25 |
| Copper (II) bromide | 7.8 | 62.5 | 0.125 |
| Copper (II) nitrate | 7.8 | 62.5 | 0.125 |
| Copper (I) acetate | 7.8 | 31.25 | 0.25 |
| Copper (II) oxychloride | 0.98 | 15.6 | 0.06 |
| Copper (II) sulphate pentahydrate | 15.6 | 125 | 0.125 |
| Copper (II)-D-gluconate | 62.5 | 125 | 0.5 |
| Copper (II) pyrithione | 0.24 | 0.49 | 0.5 |
| Copper (II) salicylate | 7.8 | 31.25 | 0.25 |
| Copper (II) aspirinate | 15.6 | 62.5 | 0.5 |
| Copper (II) silicate | 4.84 | 38.75 | 0.125 |

TABLE 4

| | | | | DDA + |
|---|---|---|---|---|
| | | DDA + | DDA + | salt & |
| | DDA | salt | lipid^{†} | lipid^{†} |
| Copper salt | (mm) | (mm) | (mm) | (mm) |
| 8-hydroxyquinoline copper (II) salt | 17.42 | 13.84 | 15.10 | 14.81 |
| | (±0.48) | | | (±0.48) |
| Copper (I) bromide | 39.21 | 29.38 | 43.92 | 43.70 |
| | (±2.08) | | | (±2.51) |
| Copper (I) iodide | 48.6 | 43.92 | 34.64 | 46.20 |
| | (±0.36) | | | (±1.48) |
| Copper (I) oxide | 48.81 | 44.85 | 37.42 | 43.49 |
| | (±0.31) | | | (±0.83) |
| Copper (II) acetate | 53.61 | 48.25 | 42.06 | 49.54 |
| | (±1.72) | | | (±1.72) |
| Copper (II) tartrate hydrate | 49.64 | 48.25 | 38.35 | 47.56 |
| | (±1.26) | | | (±2.05) |
| Copper (II) bromide | 54.86 | 47.94 | 42.37 | 49.23 |
| | (0.65±) | | | (±0.36) |
| Copper (II) nitrate | 52.46 | 50.41 | 43.61 | 48.81 |
| | (±0.79) | | | (±1.43) |
| Copper (I) acetate | 52.04 | 49.48 | 45.46 | 52.15 |
| | (±2.8) | | | (±1.26) |
| Copper (II) oxychloride | 51.21 | 54.47 | 51.65 | 52.04 |
| | (±2.39) | | | (±2.53) |
| Copper (II) sulphate pentahydrate | 49.75 | 50.28 | 44.03 | 49.96 |
| | (±1.63) | | | (±0.79) |
| Copper (II)-D-gluconate | 45.37 | 37.93 | 27.36 | 47.04 |
| | (±0.83) | | | (±0.72) |
| Copper (II) pyrithione | 56.42 | 43.21 | 22.38 | 33.37 |
| | (±0.48) | | | (±1.48) |
| Copper (II) salicylate | 47.77 | 46.08 | 41.75 | 50.69 |
| | (±0.48) | | | (±2.48) |
| Copper (II) aspirinate | 44.32 | 40.21 | 36.19 | 47.45 |
| | (±1.57) | | | (±0.36) |
| Copper (II) silicate | 45.68 | 41.44 | 33.40 | 48.81 |
| | (±1.95) | | | (±1.13) |

^{†}1% (v/v) Tween 80

TABLE 5

| | SDDA | | |
|---|---|---|---|
| | with | SDDA | |
| | TBHQ | increase | SDDA area |
| Copper salt | (mm) | (mm) | increase (%) |
| 8-hydroxyquinoline copper (II) salt | 25.34 | 7.93 | 111.6 |
| | (±1.36) | | |
| Copper (I) bromide | 54.65 | 15.44 | 94.26 |
| | (±2.91) | | |
| Copper (I) iodide | 70.08 | 21.48 | 107.92 |
| | (±2.73) | | |
| Copper (I) oxide | 50.06 | 1.25 | 5.18 |
| | (±1.43) | | |
| Copper (II) acetate | 66.96 | 13.35 | 56 |
| | (±0.54) | | |
| Copper (II) tartrate hydrate | 54.44 | 4.8 | 20.27 |
| | (±1.43) | | |
| Copper (II) bromide | 66.3 | 11.47 | 46.18 |
| | (±1.13) | | |
| Copper (II) nitrate | 68.31 | 15.85 | 69.55 |
| | (±2.66) | | |
| Copper (I) acetate | 70.71 | 18.67 | 84.62 |
| | (±3.3 1) | | |
| Copper (II) oxychloride | 59.45 | 8.24 | 34.77 |
| | (±2.17) | | |
| Copper (II) sulphate pentahydrate | 71.86 | 22.11 | 108.63 |
| | (±2.37) | | |
| Copper (II)-D-gluconate | 53.5 | 8.13 | 39.05 |
| | (±2.44) | | |
| Copper (II) pyrithione | 61.43 | 5.01 | 18.55 |
| | (±2.66) | | |
| Copper (II) salicylate | 75.61 | 27.84 | 150.52 |
| | (±1.1) | | |
| Copper (II) aspirinate | 60.80 | 16.48 | 88.19 |
| | (±0.65) | | |
| Copper (II) silicate | 54.44 | 8.76 | 42.03 |
| | (±1.13) | | |

TABLE 6

| | | | SL SDDA |
|---|---|---|---|
| | SL SDDA | SL SDDA | area |
| | with TBHQ | increase | increase |
| Copper salt | (mm) | (mm) | (%) |
| 8-hydroxyquinoline copper (II) salt | 19.82 | 5.01 | 79.03 |
| | (±0.36) | | |
| Copper (I) bromide | 56.01 | 12.31 | 64.26 |
| | (±2.05) | | |
| Copper (I) iodide | 64.77 | 18.56 | 96.51 |
| | (±0.94) | | |
| Copper (I) oxide | 51.73 | 8.24 | 41.48 |
| | (±0.48) | | |
| Copper (II) acetate | 63.62 | 14.08 | 64.92 |
| | (±0.65) | | |
| Copper (II) tartrate hydrate | 52.98 | 5.42 | 24.11 |
| | (±1.91) | | |
| Copper (II) bromide | 65.39 | 16.17 | 76.46 |
| | (±1.25) | | |
| Copper (II) nitrate | 67.27 | 18.46 | 89.94 |
| | (±1.13) | | |
| Copper (I) acetate | 65.91 | 13.77 | 59.77 |
| | (±0.18) | | |
| Copper (II) oxychloride | 54.02 | 1.98 | 7.76 |
| | (±0.96) | | |
| Copper (II) sulphate pentahydrate | 64.14 | 14.18 | 64.85 |
| | (±1.08) | | |
| Copper (II)-D-gluconate | 53.19 | 6.15 | 27.88 |
| | (±0.83) | | |
| Copper (II) pyrithione | 34.63 | 1.25 | 7.64 |
| | (±0.65) | | |
| Copper (II) salicylate | 68.94 | 18.25 | 84.98 |
| | (±1.01) | | |
| Copper (II) aspirinate | 58.93 | 11.47 | 54.20 |
| | (±0.79) | | |
| Copper (II) silicate | 54.96 | 6.15 | 26.80 |
| | (±0.36) | | |

(SL SDDA = SDDA with salt and lipid (1% (v/v) Tween 80) supplements)

These data indicate a synergistic interaction between TBHQ and the various copper salts tested, with in each case a significant increase in zone diameter over that exhibited by either compound alone. The synergies appear to be maintained even under the supplemented (salt and lipid) conditions.

### Example 6

### Activity Against S. aureus (Other Copper Salts)

A series of copper salts was tested with TBHQ using (S)DDA tests as described in Example 3. The MIC and MBC values for each copper salt were also measured as in Example 3 along with supplemented (S)DDAs conducted in the presence of salt, lipid and/or serum.

For the (S)DDA experiments, 200 μg of each test compound was loaded onto each disc with the exception of copper (II) silicate which was added at 62 μg/disc. The copper salts were dissolved in ethanol with the following exceptions: the 8-hydroxyquinoline copper (II) salt, copper (I) iodide, copper (II) pyrithione, copper (II) salicylate and copper (II) aspirinate were dissolved in DMSO; the copper (I) acetate, copper (II) acetate, copper (II)-D gluconate and copper (II) sulphate pentahydrate were dissolved in deionised water; the copper (II) tartrate hydrate was dissolved in 1 M sodium hydroxide; and the copper (II) silicate was used as a 0.62% (w/w) solution in water.

MIC and MBC experiments were performed in duplicate, supplemented DDA experiments as single replicates and SDDA experiments in triplicate.

The MIC, MBC and DDA results are shown in Tables 7 and 8 below and the SDDA results in Tables 9 (unsupplemented assays) and 10 (supplemented assays). All results are collated from a number of experiments.

TABLE 7

| | MIC | MBC | MIC/MBC |
|---|---|---|---|
| Copper salt | (μg/ml) | (μg/ml) | ratio |
| 8-hydroxyquinoline copper (II) salt | 15.6 | 62.5 | 0.25 |
| Copper (I) bromide | >250 | >250 | n/a |
| Copper (I) iodide | >250 | >250 | n/a |
| Copper (I) oxide | >250 | >250 | n/a |
| Copper (II) acetate | >250 | >250 | n/a |
| Copper (II) tartrate hydrate | >250 | >250 | n/a |
| Copper (II) bromide | >250 | >250 | n/a |
| Copper (II) nitrate | >250 | >250 | n/a |
| Copper (I) acetate | >250 | >250 | n/a |
| Copper (II) oxychloride | >250 | >250 | n/a |
| Copper (II) sulphate pentahydrate | >250 | >250 | n/a |
| Copper (II)-D-gluconate | >250 | >250 | n/a |
| Copper (II) pyrithione | 0.49 | 0.49 | 1 |
| Copper (II) salicylate | 250 | >250 | <1 |
| Copper (II) aspirinate | 125 | >250 | <0.5 |
| Copper (II) silicate | >77.5 | >77.5 | n/a |

TABLE 8

| | | | | DDA + |
|---|---|---|---|---|
| | | DDA + | DDA + | salt & |
| | DDA | salt | lipid | serum |
| Copper salt | (mm) | (mm) | (mm) | (mm) |
| 8-hydroxyquinoline copper (II) salt | 17.05 | 16.58 | 13.77 | 15.47 |
| | (±0.84) | | | (±0.00) |
| Copper (I) bromide | 0 | 0 | 0 | 0 |
| Copper (I) iodide | 0 | 0 | 0 | 0 |
| Copper (I) oxide | 0 | 0 | 0 | 0 |
| Copper (II) acetate | 0 | 0 | 0 | 0 |
| Copper (II) tartrate hydrate | 0 | 0 | 0 | 0 |
| Copper (II) bromide | 0 | 0 | 0 | 0 |
| Copper (II) nitrate | 0 | 0 | 0 | 0 |
| Copper (I) acetate | 0 | 0 | 0 | 0 |
| Copper (II) oxychloride | 0 | 0 | 0 | 0 |
| Copper (II) sulphate pentahydrate | 0 | 0 | 0 | 0 |
| Copper (II)-D-gluconate | 0 | 0 | 0 | 0 |
| Copper (II) pyrithione | 20.51 | 21.48 | 15.25 | 17.86 |
| | (±0.31) | | | (±0.47) |
| Copper (II) salicylate | 0 | 0 | 0 | 0 |
| Copper (II) aspirinate | 0 | 0 | 0 | 0 |
| Copper (II) silicate | 0 | 0 | 0 | 0 |

TABLE 9

| | SDDA | | |
|---|---|---|---|
| | with | SDDA | |
| | TBHQ | increase | SDDA area |
| Copper salt | (mm) | (mm) | increase (%) |
| 8-hydroxyquinoline copper (II) salt | 34.84 | −5.58 | −25.70 |
| | (±2.69) | | |
| Copper (I) bromide | 55.37 | 14.95 | 87.65 |
| | (±2.55) | | |
| Copper (I) iodide | 52.74 | 12.32 | 70.25 |
| | (±1.64) | | |
| Copper (I) oxide | 57.05 | 16.63 | 99.21 |
| | (±1.80) | | |
| Copper (II) acetate | 40.74 | 0.32 | 1.59 |
| | (±0.84) | | |
| Copper (II) tartrate hydrate | 13.58 | −26.84 | −88.71 |
| | (±1.58 | | |
| Copper (II) bromide | 59.26 | 18.84 | 114.95 |
| | (±1.31) | | |
| Copper (II) nitrate | 57.37 | 16.95 | 101.45 |
| | (±1.11) | | |
| Copper (I) acetate | 43.58 | 3.16 | 16.25 |
| | (±1.09) | | |
| Copper (II) oxychloride | 52.63 | 12.21 | 69.54 |
| | (±0.79) | | |
| Copper (II) sulphate pentahydrate | 55.47 | 15.05 | 88.33 |
| | (±1.11) | | |
| Copper (II)-D-gluconate | 68.32 | 27.90 | 185.69 |
| | (±5.37) | | |
| Copper (II) pyrithione | 52.45 | 19.29 | 150.13 |
| | (±1.97) | | |
| Copper (II) salicylate | 51.37 | 10.95 | 61.52 |
| | (±1.62) | | |
| Copper (II) aspirinate | 69.05 | 28.63 | 191.83 |
| | (±0.96) | | |
| Copper (II) silicate | 71.79 | 31.37 | 215.45 |
| | (±3.48) | | |

TABLE 10

| | | | SS SDDA |
|---|---|---|---|
| | SS SDDA | SS SDDA | area |
| | with TBHQ | increase | increase |
| Copper salt | (mm) | (mm) | (%) |
| 8-hydroxyquinoline copper (II) salt | 36.95 | −6.73 | −28.44 |
| | (±1.97) | | |
| Copper (I) bromide | 46.42 | 2.74 | 12.94 |
| | (±2.47) | | |
| Copper (I) iodide | 45.26 | 1.58 | 7.37 |
| | (±4.62) | | |
| Copper (I) oxide | 45.26 | 1.58 | 7.37 |
| | (±2.45) | | |
| Copper (II) acetate | 28.53 | −15.15 | −57.34 |
| | (±1.02) | | |
| Copper (II) tartrate hydrate | 10.53 | −33.15 | −94.19 |
| | (±0.36) | | |
| Copper (II) bromide | 44.42 | 0.74 | 3.42 |
| | (±1.74) | | |
| Copper (II) nitrate | 40.74 | −2.94 | −13.01 |
| | (±4.96) | | |
| Copper (I) acetate | 30.32 | −13.36 | −51.82 |
| | (±3.52) | | |
| Copper (II) oxychloride | 43.89 | 0.21 | 0.96 |
| | (±1.38) | | |
| Copper (II) sulphate pentahydrate | 46.00 | 2.32 | 10.90 |
| | (±2.03) | | |
| Copper (II)-D-gluconate | 53.37 | 9.69 | 49.29 |
| | (±0.32) | | |
| Copper (II) pyrithione | 41.43 | 9.29 | 66.12 |
| | (±0.88) | | |
| Copper (II) salicylate | 45.68 | 2.00 | 9.37 |
| | (±1.31) | | |
| Copper (II) aspirinate | 56.95 | 13.27 | 69.99 |
| | (±10.61) | | |
| Copper (II) silicate | 57.26 | 13.58 | 71.85 |
| | (±4.00) | | |

(SS SDDA = SDDA with salt and serum supplements)

These data indicate that many of the copper salts tested have relatively low or no activity, on their own, against S. aureus ATCC 29213. Surprisingly, however, they appear able to potentiate the activity of the quinone against the organism. This effect is in many cases still observed under the supplemented (salt and serum) conditions.

### Example 7

### Activity Against Other Propionibacterium spp

The activity (MIC by agar dilution and DDA) of copper (II) sulphate pentahydrate (CSPH) alone and in combination with TBHQ was determined against a panel of different propionibacterium strains. For (S)DDA experiments 200 μg of each test compound was added per disc. The TBHQ was dissolved in ethanol and the copper (II) sulphate pentahydrate in deionised water. MIC experiments were performed in duplicate, DDA experiments as single replicates only. SDDA experiments were conducted in triplicate.

The MIC and DDA results for the copper salt are shown in Table 11, and SDDA results with TBHQ in Table 12. The tables indicate the resistance phenotype for each of the test species/strains.

TABLE 11

| | | | | | CSPH |
|---|---|---|---|---|---|
| | | | CSPH | CSPH | DDA + |
| | Resistance | CSPH MIC | DDA | DDA + | lipid^{†} |
| Test organism | phenotype | (μg/ml) | (mm) | salt (mm) | (mm) |
| P. granulosum NCTC 11865 | None | 7.8 | 48.34 | 48.34 | 41.65 |
| P. acnes PRP-002 | Tet/MLS | 7.8 | 49.25 | 50.77 | 47.13 |
| P. acnes PRP-003 | Tet | 7.8 | 52.60 | 51.99 | 45.91 |
| P. acnes PRP-004 | Tet | 7.8 | 51.08 | 52.60 | 45.61 |
| P. granulosum PRP-005 | MLSK | 62.5 | 24.02 | 29.19 | 24.32 |
| P. granulosum PRP-006 | MLS | 62.5 | 31.62 | 37.09 | 30.71 |
| P. acnes PRP-007 | Clin | 7.8 | 56.55 | 55.03 | 48.95 |
| P. acnes PRP-008 | Clin | 7.8 | 52.29 | 51.99 | 48.65 |
| P. acnes PRP-010 | MLSK | 7.8 | 63.24 | 62.33 | 46.82 |
| P. acnes PRP-017 | MLS | 7.8 | 51.38 | 56.85 | 55.03 |
| P. granulosum PRP-019 | MLSK | 62.5 | 22.80 | 25.84 | 24.02 |
| P. granulosum PRP-021 | MLS | 62.5 | 27.67 | 32.84 | 28.28 |
| P. acnes PRP-023 | MLSK | 7.8 | 47.13 | 51.38 | 47.43 |
| P. acnes PRP-026 | MLS | 7.8 | 51.08 | 52.90 | 53.21 |
| P. acnes PRP-039 | Tet/MLS | 7.8 | 53.51 | 61.72 | 59.29 |
| P. granulosum PRP-043 | MLS | 7.8 | 46.82 | 50.17 | 41.35 |
| P. granulosum PRP-044 | MLS | 62.5 | 27.36 | 31.92 | 26.45 |
| P. acnes PRP-046 | None | 7.8 | 53.81 | 63.54 | 60.81 |
| P. acnes PRP-053 | Tet/MLS | 7.8 | 43.78 | 54.42 | 51.08 |
| P. granulosum PRP-055 | None | 62.5 | 27.67 | 28.88 | 27.97 |
| P. acnes PRP-059 | MLS | 7.8 | 48.04 | 55.33 | 48.95 |
| P. acnes PRP-068 | Ery | 7.8 | 52.90 | 54.73 | 49.56 |
| P. acnes PRP-101 | Tet/MLS | 7.8 | 45.30 | 53.21 | 55.03 |
| P. acnes PRP-102 | Tet/MLS | 7.8 | 46.52 | 49.25 | 50.77 |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]
^{†}1% (v/v) triolein

TABLE 12

| | | | | | | SDDA |
|---|---|---|---|---|---|---|
| | | DDA | DDA | SDDA | SDDA | area |
| | Resistance | TBHQ | CSPH | TBHQ + | increase | increase |
| Test organism | phenotype | (mm) | (mm) | CSPH | (mm) | (%) |
| P. granulosum NCTC 11865 | None | 10.45 | 53.32 | 60.01 | 6.69 | 26.67 |
| | | (±0.48) | (±0.83) | (±0.18) | | |
| P. acnes PRP-002 | Tet/MLS | 10.56 | 57.92 | 65.55 | 7.63 | 28.09 |
| | | (±0.48) | (±0.48) | (±2.57) | | |
| P. acnes PRP-003 | Tet | 18.61 | 50.18 | 68.37 | 18.19 | 85.64 |
| | | (±1.19) | (±2.72) | (±2.73) | | |
| P. acnes PRP-004 | Tet | 13.80 | 44.85 | 65.76 | 20.91 | 114.97 |
| | | (±0.31) | (±0.31) | (±1.99) | | |
| P. granulosum PRP-005 | MLSK | 15.05 | 22.48 | 35.44 | 12.96 | 148.61 |
| | | (±1.63) | (±0.65) | (±0.83) | | |
| P. granulosum PRP-006 | MLS | 9.93 | 27.91 | 40.98 | 13.07 | 115.55 |
| | | (±1.48) | (±0.31) | (±0.65) | | |
| P. acnes PRP-007 | Clin | 8.78 | 40.35 | 61.47 | 21.12 | 132.05 |
| | | (±0.31) | (±0.79) | (±1.75) | | |
| P. acnes PRP-008 | Clin | 13.90 | 43.70 | 70.25 | 26.55 | 158.46 |
| | | (±1.31) | (±1.10) | (±2.26) | | |
| P. acnes PRP-010 | MLSK | 8.68 | 46.73 | 71.40 | 24.67 | 133.47 |
| | | (±0.48) | (±3.09) | (±1.55) | | |
| P. acnes PRP-017 | MLS | 9.93 | 46.31 | 64.92 | 18.61 | 96.51 |
| | | (±0.65) | (±1.58) | (±1.13) | | |
| P. granulosum PRP-019 | MLSK | 0.00 | 24.15 | 38.68 | 14.53 | 156.55 |
| | | (±0.00) | (±0.31) | (±0.72) | | |
| P. granulosum PRP-021 | MLS | 8.57 | 29.59 | 42.86 | 13.28 | 109.89 |
| | | (±0.18) | (±0.48) | (±0.36) | | |
| P. acnes PRP-023 | MLSK | 11.92 | 60.95 | 69.00 | 8.05 | 28.16 |
| | | (±1.57) | (±1.31) | (±1.09) | | |
| P. acnes PRP-026 | MLS | 11.29 | 56.14 | 67.22 | 11.08 | 43.38 |
| | | (±0.83) | (±1.25) | (±0.72) | | |
| P. acnes PRP-039 | Tet/MLS | 17.04 | 56.35 | 74.44 | 18.09 | 74.49 |
| | | (±2.27) | (±2.67) | (±0.48) | | |
| P. granulosum PRP-043 | MLS | 10.77 | 56.56 | 62.41 | 5.85 | 21.77 |
| | | (±1.84) | (±0.96) | (±0.63) | | |
| P. granulosum PRP-044 | MLS | 0.00 | 27.81 | 40.56 | 12.75 | 112.77 |
| | | (±0.00) | (±0.65) | (±0.48) | | |
| P. acnes PRP-046 | None | 14.22 | 50.29 | 76.84 | 26.55 | 133.50 |
| | | (±1.78) | (±2.83) | (±0.83) | | |
| P. acnes PRP-053 | Tet/MLS | 9.93 | 50.18 | 62.00 | 11.81 | 52.63 |
| | | (±0.79) | (±0.83) | (±1.27) | | |
| P. granulosum PRP-055 | None | 10.04 | 31.78 | 44.95 | 13.17 | 100.07 |
| | | (±0.31) | (±0.96) | (±1.01) | | |
| P. acnes PRP-059 | MLS | 10.87 | 50.70 | 63.46 | 12.75 | 56.64 |
| | | (±0.91) | (±0.65) | (±1.01) | | |
| P. acnes PRP-068 | Ery | 12.55 | 51.12 | 69.94 | 18.82 | 87.17 |
| | | (±0.83) | (±1.13) | (±0.83) | | |
| P. acnes PRP-101 | Tet/MLS | 14.01 | 43.49 | 74.02 | 30.53 | 189.65 |
| | | (±2.98) | (±2.40) | (±0.94) | | |
| P. acnes PRP-102 | Tet/MLS | 9.10 | 51.65 | 67.64 | 16.00 | 71.54 |
| | | (±0.31) | (±2.91) | (±1.01) | | |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

These data indicate that copper (II) sulphate pentahydrate demonstrates an excellent level of activity against all propionibacterium strains tested. This activity appears to be maintained even under the supplemented (salt and lipid) conditions. However, the majority of P. granulosum strains appear to exhibit a degree of resistance to the copper salt as demonstrated by increased MIC levels and smaller DDA zones of inhibition.

When the copper salt and the quinone are combined, however, the synergistic interaction initially observed with P. acnes NCTC 737 was similarly observed with all of the propionibacterium strains tested as part of this panel.

### Example 8

### Activity Against P. acnes (Other Quinones)

A number of different quinones was tested with both copper (II) sulphate pentahydrate (CSPH) and copper (II) silicate (CSL) against P. acnes NCTC 737 using (S)DDA tests as described in Example 1.

For the (S)DDA experiments, 200 μg of each test compound was loaded onto each disc, with the exception of copper (II) silicate (62 μg/disc) and 2-t-butyl-1,4-benzoquinone (100 μg/disc). The quinones were dissolved in DMSO and the copper (II) sulphate pentahydrate in deionised water, whilst the copper (II) silicate was used as a 0.62% (w/w) solution in water.

All the quinones were sourced from Thermo Fisher Scientific, UK with the exception of 2-t-butyl-1,4-benzoquinone (Sigma-Aldrich) and 2-ethyl-p-hydroquinone (Apin Chemicals Ltd, UK).

The DDA results are shown in Table 13 below and the SDDA results in Tables 14 and 15 (unsupplemented assays) and 16 and 17 (supplemented assays).

TABLE 13

| | | DDA + salt & |
|---|---|---|
| Test compound | DDA (mm) | lipid^{†} (mm) |
| Copper (II) sulphate pentahydrate | 42.05 (±1.16) | 49.54 (±1.34) |
| Copper (II) silicate | 40.82 (±0.17) | 42.56 (±2.18) |
| 2-t-butyl-1,4-benzoquinone | 35.18 (±0.47) | 45.13 (±1.45) |
| 2-methylhydroquinone | 13.64 (±13.64) | 35.18 (±4.06) |
| 2-methyl-p-benzoquinone | 33.74 (±1.52) | 35.18 (±1.24) |
| 2-chloro-1,4-benzoquinone | 22.15 (±0.53) | 24.31 (±0.81) |
| 2-ethylhydroquinone | 9.95 (±0.35) | 33.74 (±1.69) |

^{†}1% (v/v) triolein

TABLE 14

| | | | SDDA |
|---|---|---|---|
| | | SDDA | area |
| | SDDA with | increase | increase |
| Quinone | CSPH (mm) | (mm) | (%) |
| 2-t-butyl-1,4-benzoquinone | 53.44 (±0.47) | 11.39 | 61.51 |
| 2-methyl-p-hydroquinone | 46.87 (±0.64) | 4.82 | 24.24 |
| 2-methyl-p-benzoquinone | 49.54 (±0.81) | 7.49 | 38.80 |
| 2-chloro-1,4-benzoquinone | 47.28 (±1.39) | 5.23 | 26.42 |
| 2-ethyl-p-hydroquinone | 50.46 (±1.11) | 8.41 | 44.00 |

TABLE 15

| | | | SDDA |
|---|---|---|---|
| | | SDDA | area |
| | SDDA with CSL | increase | increase |
| Quinone | (mm) | (mm) | (%) |
| 2-t-butyl-1,4-benzoquinone | 52.20 (±0.78) | 11.39 | 63.59 |
| 2-methyl-p-hydroquinone | 45.54 (±1.11) | 4.72 | 24.46 |
| 2-methyl-p-benzoquinone | 49.64 (±0.99) | 8.82 | 47.88 |
| 2-chloro-1,4-benzoquinone | 45.85 (±0.31) | 5.03 | 26.16 |
| 2-ethyl-p-hydroquinone | 47.08 (±1.60) | 6.26 | 33.02 |

TABLE 16

| | | SL SDDA | SL SDDA |
|---|---|---|---|
| | SL SDDA with | increase | area |
| Quinone | CSPH (mm) | (mm) | increase (%) |
| 2-t-butyl-1,4-benzoquinone | 52.10 (±2.56) | 2.56 | 10.60 |
| 2-methyl-p-hydroquinone | 53.74 (±1.75) | 4.20 | 17.67 |
| 2-methyl-p-benzoquinone | 53.95 (±1.16) | 4.41 | 18.59 |
| 2-chloro-1,4-benzoquinone | 50.46 (±2.15) | 0.92 | 3.75 |
| 2-ethyl-p-hydroquinone | 52.21 (±0.18) | 2.67 | 11.07 |

(SL SDDA = SDDA with salt and lipid (1% (v/v) triolein) supplements)

TABLE 17

| | | SL SDDA | SL SDDA |
|---|---|---|---|
| | SL SDDA with | increase | area |
| Quinone | CSL (mm) | (mm) | increase (%) |
| 2-t-butyl-1,4-benzoquinone | 56.41 (±2.58) | 11.28 | 56.24 |
| 2-methyl-p-hydroquinone | 49.54 (±0.81) | 6.98 | 35.49 |
| 2-methyl-p-benzoquinone | 49.85 (±3.20) | 7.29 | 37.19 |
| 2-chloro-1,4-benzoquinone | 48.82 (±0.47) | 6.26 | 31.58 |
| 2-ethyl-p-hydroquinone | 48.82 (±0.47) | 6.26 | 31.58 |

(SL SDDA = SDDA with salt and lipid (1% (v/v) triolein) supplements)

These data demonstrate synergistic antimicrobial activity against P. acnes NCTC 737 for a range of different benzo- and hydroquinones with copper salts. On the whole these synergies are retained under the supplemented conditions.

### Example 9

### Activity Against S. aureus (Other Guinones)

Two further quinones, 2-methyl- and 2-ethyl-p-hydroquinone, were tested with both copper (II) sulphate pentahydrate (CSPH) and copper (II) silicate (CSL) against S. aureus ATCC 29213 using (S)DDA tests as described in Example 3.

For the (S)DDA experiments, 200 μg of each test compound was loaded onto each disc with the exception of copper (II) silicate which was added at 62 μg/disc. The quinones were dissolved in DMSO and the copper (II) sulphate pentahydrate in deionised water, whilst the copper (II) silicate was used as a 0.62% (w/w) solution in water. All (S)DDA experiments were conducted in triplicate.

The DDA results are shown in Table 18 below and the SDDA results in Tables 19 and 20 (unsupplemented assays) and 21 and 22 (supplemented assays).

TABLE 18

| | | DDA + salt & |
|---|---|---|
| Test compound | DDA (mm) | serum (mm) |
| Copper (II) sulphate pentahydrate | 0.00 (±0.00) | 0.00 (±0.00) |
| Copper (II) silicate | 0.00 (±0.00) | 0.00 (±0.00) |
| 2-methyl-p-hydroquinone | 25.77 (±0.36) | 26.91 (±0.00) |
| 2-ethyl-p-hydroquinone | 24.12 (±0.00) | 25.26 (±1.29) |

TABLE 19

| | | | SDDA |
|---|---|---|---|
| | | SDDA | area |
| | SDDA with | increase | increase |
| Quinone | CSPH (mm) | (mm) | (%) |
| 2-methyl-p-hydroquinone | 40.00 (±0.47) | 14.23 | 140.93 |
| 2-ethyl-p-hydroquinone | 39.07 (±0.72) | 14.74 | 157.87 |

TABLE 20

| | | | SDDA |
|---|---|---|---|
| | | SDDA | area |
| | SDDA with CSL | increase | increase |
| Quinone | (mm) | (mm) | (%) |
| 2-methyl-p-hydroquinone | 37.52 (±0.36) | 11.76 | 112.09 |
| 2-ethyl-p-hydroquinone | 38.56 (±2.06) | 14.23 | 151.18 |

TABLE 21

| | | SS SDDA | SS SDDA |
|---|---|---|---|
| | SS SDDA with | increase | area |
| Quinone | CSPH (mm) | (mm) | increase (%) |
| 2-methyl-p-hydroquinone | 35.57 (±0.62) | 8.66 | 74.72 |
| 2-ethyl-p-hydroquinone | 32.78 (±0.31) | 7.52 | 68.40 |

(SS SDDA = SDDA with salt and serum supplements)

TABLE 22

| | | SS SDDA | SS SDDA |
|---|---|---|---|
| | SS SDDA with | increase | area |
| Quinone | CSL (mm) | (mm) | increase (%) |
| 2-methyl-p-hydroquinone | 37.11 (±1.11) | 10.20 | 90.18 |
| 2-ethyl-p-hydroquinone | 30.00 (±2.47) | 4.74 | 41.05 |

(SS SDDA = SDDA with salt and serum supplements)

These data demonstrate that a synergistic antimicrobial interaction against S. aureus ATCC 29213 can be observed not only for TBHQ with copper salts but also for other benzo- and hydroquinones with copper salts. The synergies are retained under the supplemented conditions.

### Example 10

### Topical Anti-Acne Formulations

The results from Examples 1, 2, 5, 7 and 8 show that the combination of a quinone and a copper salt can be an effective antimicrobial agent, in particular against the bacteria associated with acne, with a synergistic impact on the antimicrobial activity of the combination compared to that of the individual compounds alone. This can be of use in preparing antimicrobial formulations, in particular for topical application to the skin, for either prophylactic or therapeutic use in any context where such bacteria are thought to be involved as possible sources of infection.

Even in cases where a combination of a quinone and a copper salt has an additive, as opposed to synergistic, antimicrobial activity compared to that of the individual compounds, this can be of considerable benefit when preparing formulations for topical use. One of the compounds may be used to replace a proportion of the other, thus lowering any side effects and/or other undesirable properties of the combination without undue loss of antimicrobial activity.

A topical formulation for use in treating acne may for example be prepared by combining a quinone, in particular an alkyl-substituted hydroquinone such as TBHQ, with a copper salt such as copper salicylate or copper aspirinate, in a suitable fluid vehicle and optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman R et al, “Skin penetration and distribution of polymeric nanoparticles”, J. Controlled Release 2004; 99: 53-62.

The formulation may be prepared and administered using known techniques. It may for example take the form of a cream, lotion or gel.

The concentrations of the two active agents may be in the ranges described above, and will be determined based on the intended use of the formulation, its intended mode of administration and the activities of the particular chosen active agents.

### Example 11

### Topical Anti-Staphylococcal Formulation

A formulation for use against staphylococci such as S. aureus may be prepared by combining a quinone, in particular an alkyl-substituted hydroquinone such as TBHQ, with a copper salt such as copper gluconate or copper aspirinate, in a similar manner to that described for the anti-acne formulation. The ingredients may in this case be formulated as a spray, for instance for application to work surfaces or surgical instruments; as a cleansing gel or lotion for instance for hand washing; as a nasal spray for application to the anterior nares or in many other appropriate forms. Such a formulation may in particular be used prophylactically, eg, to reduce the risk of outbreaks of MRSA or similar infections.

### Example 12

### Activity Against Propionibacterium spp—Copper 8-HO

It can be seen from Example 5 above that copper 8-HQ is highly active as an antibacterial agent against P. acnes NCTC 737, its activity comparing favourably with those of other copper salts tested in the same example. This indicates its likely activity as an anti-acne agent.

The antibacterial activity is maintained in the presence of both salt and lipid, indicating the suitability of the compound for the topical treatment of acne.

The activity of the salt (MIC by agar dilution and DDA) was also determined against a panel of different propionibacterium strains. Supplemented DDA experiments were also carried out. For the DDA experiments 200 μg of the copper 8-HQ was loaded onto each disc. DMSO was used as the diluting solvent. MIC experiments were performed in duplicate and DDA experiments as single replicates only.

The results are shown in Table 23 below, which also indicates the resistance phenotype for each of the test strains.

TABLE 23

| | | Cu-8HQ | | | DDA + |
|---|---|---|---|---|---|
| | Resistance | MIC | | DDA + | lipid^{†} |
| Test organism | phenotype | (μg/ml) | DDA (mm) | salt (mm) | (mm) |
| P. acnes NCTC 737 | None | 1.95 | 22.27 | 26.98 | 22.59 |
| P. granulosum NCTC 11865 | None | 1.95 | 23.22 | 24.78 | 23.84 |
| P. acnes PRP-002 | Tet/MLS | 1.95 | 20.08 | 27.92 | 25.41 |
| P. acnes PRP-003 | Tet | 1.95 | 20.39 | 22.59 | 24.78 |
| P. acnes PRP-004 | Tet | 3.9 | 19.76 | 22.90 | 24.47 |
| P. granulosum PRP-005 | MLSK | 3.9 | 19.14 | 19.76 | 20.08 |
| P. granulosum PRP-006 | MLS | 3.9 | 21.96 | 28.55 | 21.65 |
| P. acnes PRP-007 | Clin | 1.95 | 19.14 | 21.02 | 25.41 |
| P. acnes PRP-008 | Clin | 1.95 | 20.08 | 22.27 | 22.90 |
| P. acnes PRP-010 | MLSK | 1.95 | 21.96 | 21.96 | 25.41 |
| P. acnes PRP-017 | MLS | 3.9 | 19.76 | 22.59 | 24.78 |
| P. granulosum PRP-019 | MLSK | 3.9 | 19.76 | 19.45 | 20.39 |
| P. granulosum PRP-021 | MLS | 3.9 | 21.33 | 26.67 | 20.71 |
| P. acnes PRP-023 | MLSK | 1.95 | 22.27 | 22.27 | 25.73 |
| P. acnes PRP-026 | MLS | 1.95 | 21.96 | 23.84 | 26.67 |
| P. acnes PRP-039 | Tet/MLS | 1.95 | 21.02 | 30.12 | 25.41 |
| P. granulosum PRP-043 | MLS | 1.95 | 23.53 | 29.49 | 22.59 |
| P. granulosum PRP-044 | MLS | 3.9 | 20.08 | 21.96 | 19.14 |
| P. acnes PRP-046 | None | 1.95 | 20.08 | 21.33 | 23.84 |
| P. acnes PRP-053 | Tet/MLS | 1.95 | 24.16 | 22.27 | 23.53 |
| P. granulosum PRP-055 | None | 3.9 | 17.25 | 16.31 | 18.20 |
| P. acnes PRP-059 | MLS | 1.95 | 22.27 | 23.84 | 24.47 |
| P. acnes PRP-068 | Ery | 1.95 | 19.76 | 21.02 | 22.27 |
| P. acnes PRP-101 | Tet/MLS | 1.95 | 20.39 | 26.98 | 24.78 |
| P. acnes PRP-102 | Tet/MLS | 1.95 | 21.96 | 22.59 | 21.96 |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]
^{†}1% (v/v) triolein

The high activity of the copper 8-HQ against a wide range of propionibacterial species and strains, including under supplemented conditions, is of particular note. Such wide-ranging activity is not exhibited by all copper salts, for example copper (II) sulphate pentahydrate (see Example 5), copper (II) silicate and copper nanopowder. The compound's activity against strains of P. granulosum is of particular value in the context of anti-acne formulations, since these bacteria tend to be implicated in more severe forms of acne.

### Example 13

### Activity Against Staphylococci—Copper 8-HO

It can be seen from Example 6 above that copper 8-HQ is active as an antibacterial agent against S. aureus ATCC 29213, its activity comparing very favourably with those of other copper salts tested in the same example. The activity is also maintained in the presence of both salt and serum.

The activity of the salt was also determined against a panel of different staphylococcal strains, including some with known antibiotic resistance. MIC, MBC and DDA assays were carried out as described above for each of the strains, including determining the effect of the presence of salt and serum.

For DDA experiments 200 μg of the test compound was added per disc. DMSO was used as the diluting solvent. All experiments were performed in triplicate.

The MIC and MBC results are shown in Table 24 below and the DDA results in Table 25. The tables also indicate the resistance phenotype for each of the test organisms.

TABLE 24

| | | Cu-8HQ | Cu-8HQ | |
|---|---|---|---|---|
| | | MIC | MBC | MIC/MBC |
| Test organism | Resistance phenotype | (μg/ml) | (μg/ml) | ratio |
| Staphylococcus | ND | 7.8 | 7.8 | 1 |
| simulans ATCC 27848 | | | | |
| Staphylococcus | ND | 7.8 | 7.8/15.6 | 1/0.5 |
| xylosus ATCC 29971 | | | | |
| Staphylococcus cohnii | ND | 7.8 | 7.8/15.6 | 1/0.5 |
| ATCC 29974 | | | | |
| Staphylococcus | ND | 7.8 | 7.8/15.6 | 1/0.5 |
| haemolyticus ATCC | | | | |
| 29970 | | | | |
| Staphylococcus | ND | 3.9/7.8 | 7.8/15.6 | 0.25/0.5/1 |
| warneri ATCC 27836 | | | | |
| Staphylococcus capitis | ND | 3.9/7.8 | 15.6 | 0.25/0.5 |
| ATCC 27840 | | | | |
| Staphylococcus | ND | 7.8 | 31.25 | 0.25 |
| hominis ATCC 27844 | | | | |
| Staphylococcus | ND | 3.9 | 3.9/7.8 | 1/0.5 |
| auricularis ATCC | | | | |
| 33753 | | | | |
| Staphylococcus aureus | ND | 3.9 | 7.8 | 0.5 |
| ATCC 12600 | | | | |
| S. aureus ATCC | ND | 3.9 | 3.9/7.8 | 1/0.5 |
| 12600-U | | | | |
| S. aureus ATCC 12601 | ND | 3.9/7.8 | 7.8/15.6 | 0.25/0.5/1 |
| S. aureus ATCC 12602 | ND | 3.9 | 7.8 | 0.5 |
| S. aureus ATCC 12604 | ND | 7.8 | 7.8 | 1 |
| S. aureus ATCC 12605 | ND | 7.8 | 7.8/15.6 | 1/0.5 |
| S. aureus ATCC 12606 | ND | 3.9/7.8 | 7.8/15.6 | 0.25/0.5/1 |
| S. aureus ATCC 12607 | ND | 3.9 | 3.9/7.8 | 1/0.5 |
| S. aureus ATCC | ND | 3.9/7.8 | 7.8/15.6 | 0.25/0.5/1 |
| 29213 | | | | |
| S. aureus ATCC | ND | 7.8 | 7.8/15.6 | 1/0.5 |
| 25923 | | | | |
| S. aureus CPHL | Met/β-lactams* | 3.9/7.8 | 7.8 | 0.5/1 |
| EMRSA 15 | | | | |
| S. aureus CPHL | Met/β-lactams* | 3.9/7.8 | 3.9/7.8 | 1/0.5 |
| EMRSA 16 | | | | |
| S. aureus CPHL | Met/β-lactams* | 3.9 | 3.9/7.8 | 1/0.5 |
| EMRSA 17 | | | | |
| S. aureus CPHL VISA | Van* (intermediate) | 3.9 | 3.9/7.8 | 1/0.5 |
| Mu3 | | | | |
| S. aureus CPHL VISA | Van* (intermediate) | 1.95/3.9 | 3.9/7.8 | 0.25/0.5/1 |
| Mu50 | | | | |
| S. aureus CPHL GISA | Van/Tec* (intermediate) | 3.9 | 7.8 | 0.5 |
| HO41340156 | | | | |
| S. saprophyticus | ND | 1.95/3.9 | 1.95/3.9 | 1/0.5 |
| NCTC 7292 | | | | |
| S. epidermidis | ND | 1.95/3.9 | 7.8 | 0.25/0.5 |
| (NCTC 11047 | | | | |

[Abbreviations: American Type Culture Collection (ATCC), Central Public Health Laboratory UK (CPHL), National Collection of Type Cultures (NCTC), Methicillin (Met), Vancomycin (Van), Teicoplanin (Tec), not determined (ND), epidemic methicillin resistant S. aureus (EMRSA), vancomycin intermediate S. aureus (VISA), glycopeptide resistant S. aureus (GISA).]
*Other uncharacterised antibiotic resistances may be present.

TABLE 25

| | | | | DDA + |
|---|---|---|---|---|
| | Resistance | DDA | DDA + | serum |
| Test organism | phenotype | (mm) | salt (mm) | (mm) |
| Staphylococcus | ND | 18.35 | 18.77 | 17.21 |
| simulans ATCC 27848 | | (±0.54) | (±0.18) | (±0.95) |
| Staphylococcus | ND | 18.77 | 18.77 | 15.97 |
| xylosus ATCC 29971 | | (±0.72) | (±0.36) | (±0.18) |
| Staphylococcus cohnii | ND | 21.36 | 20.63 | 19.18 |
| ATCC 29974 | | (±0.18) | (±0.65) | (±1.53) |
| Staphylococcus | ND | 18.35 | 18.25 | 15.45 |
| haemolyticus ATCC | | (±0.62) | (±0.65) | (±0.36) |
| 29970 | | | | |
| Staphylococcus | ND | 20.63 | 19.91 | 16.80 |
| warneri ATCC 27836 | | (±0.18) | (±0.31) | (±0.54) |
| Staphylococcus capitis | ND | 17.83 | 18.77 | 16.07 |
| ATCC 27840 | | (±0.65) | (±0.48) | (±0.18) |
| Staphylococcus | ND | 18.15 | 18.66 | 16.38 |
| hominis ATCC 27844 | | (±0.48) | (±0.62) | (±0.36) |
| Staphylococcus | ND | 22.19 | 20.84 | 18.98 |
| auricularis ATCC | | (±0.65) | (±0.31) | (±0.62) |
| 33753 | | | | |
| Staphylococcus aureus | ND | 19.29 | 18.77 | 16.28 |
| ATCC 12600 | | (±0.31) | (±0.36) | (±0.18) |
| S. aureus ATCC | ND | 19.49 | 18.56 | 16.80 |
| 12600-U | | (±0.18) | (±0.48) | (±1.12) |
| S. aureus ATCC 12601 | ND | 18.04 | 17.52 | 16.28 |
| | | (±0.31) | (±0.36) | (±0.48) |
| S. aureus ATCC 12602 | ND | 18.66 | 17.73 | 15.66 |
| | | (±0.62) | (±0.00) | (±0.18) |
| S. aureus ATCC 12604 | ND | 17.73 | 17.63 | 15.35 |
| | | (±0.00) | (±0.18) | (±0.18) |
| S. aureus ATCC 12605 | ND | 17.94 | 17.52 | 14.52 |
| | | (±0.18) | (±0.36) | (±0.18) |
| S. aureus ATCC 12606 | ND | 18.46 | 17.42 | 14.83 |
| | | (±0.48) | (±0.31) | (±0.18) |
| S. aureus ATCC 12607 | ND | 20.43 | 19.18 | 16.28 |
| | | (±0.48) | (±0.18) | (±1.30) |
| S. aureus ATCC | ND | 18.04 | 17.73 | 15.04 |
| 29213 | | (±0.82) | (±0.31) | (±0.18) |
| S. aureus ATCC | ND | 18.77 | 18.15 | 15.04 |
| 25923 | | (±0.48) | (±0.48) | (±0.18) |
| S. aureus CPHL | Met/β-lactams* | 19.18 | 17.52 | 14.93 |
| EMRSA 15 | | (±0.18) | (±0.36) | (±0.31) |
| S. aureus CPHL | Met/β-lactams* | 20.32 | 19.91 | 16.90 |
| EMRSA 16 | | (±0.48) | (±0.00) | (±0.90) |
| S. aureus CPHL | Met/β-lactams* | 21.88 | 20.74 | 17.94 |
| EMRSA 17 | | (±0.48) | (±0.36) | (±1.09) |
| S. aureus CPHL VISA | Van* | 20.43 | 19.39 | 18.04 |
| Mu3 | (intermediate) | (±0.48) | (±0.36) | (±0.31) |
| S. aureus CPHL VISA | Van* | 20.84 | 20.22 | 17.52 |
| Mu50 | (intermediate) | (±0.00) | (±0.31) | (±1.00) |
| S. aureus CPHL GISA | Van/Tec* | 18.77 | 17.63 | 15.14 |
| HO41340156 | (intermediate) | (±0.18) | (±0.36) | (±0.18) |
| S. saprophyticus | ND | 20.32 | 20.53 | 17.42 |
| NCTC 7292 | | (±0.48) | (±0.31) | (±0.31) |
| S. epidermidis | ND | 21.15 | 20.95 | 18.46 |
| (NCTC 11047 | | (±0.31) | (±0.18) | (±0.36) |

[Abbreviations: American Type Culture Collection (ATCC), Central Public Health Laboratory UK (CPHL), National Collection of Type Cultures (NCTC), Methicillin (Met), Vancomycin (Van), Teicoplanin (Tec), not determined (ND), epidemic methicillin resistant S. aureus (EMRSA), vancomycin intermediate S. aureus (VISA), glycopeptide resistant S. aureus (GISA).]
*Other uncharacterised antibiotic resistances may be present.

These data demonstrate the high activity of copper 8-HQ against a wide range of staphylococcal strains, including under supplemented conditions. Again this contrasts with the virtually complete inactivity of other copper salts against the organism, as demonstrated in Example 6. Retention of activity in the presence of both salt and serum indicates the suitability of the compound for the topical treatment of staphylococcal infections.

### Example 14

### Topical Anti-Acne Formulations

The results from Example 12 show that copper 8-hydroxyquinoline can be an effective antimicrobial agent, in particular against the bacteria associated with acne. This can be of use in preparing antibacterial formulations, in particular for topical application to the skin, for either prophylactic or therapeutic use in any context where such bacteria are thought to be involved as possible sources of infection. More specifically, it can be of use in preparing anti-acne formulations, again suitably for topical use.

A topical formulation for use in treating acne may for example be prepared by formulating copper 8-HQ in a suitable fluid vehicle, optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman R et al, “Skin penetration and distribution of polymeric nanoparticles”, J. Controlled Release 2004; 99: 53-62.

The formulation may be prepared and administered using known techniques. It may for example take the form of a cream, lotion, ointment or gel.

The concentration of the copper 8-HQ may be in the ranges described above, and will be determined based on its antibacterial activity and the intended use of the formulation.

### Example 15

### Topical Anti-Staphylococcal Formulation

A formulation for use against staphylococci such as S. aureus may be prepared by formulating copper 8-HQ in a suitable fluid vehicle, in a similar manner to that described for the anti-acne formulation of Example 14. The ingredients may in this case be formulated as a spray, for instance for application to work surfaces or surgical instruments; as a cleansing gel or lotion for instance for hand washing; as a nasal spray for application to the anterior nares or in many other appropriate forms. Such a formulation may in particular be used prophylactically, eg, to reduce the risk of outbreaks of MRSA or similar infections.

1. An antimicrobial formulation comprising (a) a mono- or di-alkyl-substituted benzoquinone, a mono- or di-alkyl-substituted hydroquinone or a mixture thereof, wherein the alkyl group(s) are attached to carbon atom(s) of the cyclohexyl ring, and (b) a copper salt.

2. (canceled)

3. A formulation according to claim 1, which is suitable for topical application to the skin.

4. (canceled)

5. A formulation according to claim 14, wherein the two C═O groups or C—OH groups of the benzoquinone or hydroquinone are positioned para to one another.

6-10. (canceled)

11. A formulation according to claim 1, wherein the quinone is TBHQ.

12. (canceled)

13. A formulation according to claim 1, wherein the copper salt is selected from copper carboxylates, copper halides, copper sulphadiazine, copper sulphate, copper nitrate, copper carbonate, copper gluconate, copper oxide, copper peptides, copper silicates, copper salts of quinolines and their derivatives, copper pyrithione and other copper salts of pyridine thiols, and mixtures thereof.

14-17. (canceled)

18. A formulation according to claim 1, wherein the concentration of the quinone is 0.5% w/v or greater.

19. A formulation according to claim 1, wherein the concentration of the copper salt is 0.1% w/v or greater.

20. A formulation according to claim 1, wherein the weight ratio of the quinone to the copper salt is from 50:1 to 1:1.

21. (canceled)

22. A formulation according to claim 1, which additionally comprises one or more agents selected from anti-acne agents, keratolytics, comedolytics, anti-inflammatories, anti-proliferatives, antibiotics, antiandrogens, sebostatic agents, anti-pruritics, immunomodulators, agents which promote wound healing, sunscreens, moisturisers, emollients and mixtures thereof.

23. A formulation according to claim 1, which is in the form of a cream, paste, gel, ointment, lotion, foam or other viscous or semiviscous fluid.

24. A formulation according to claim 1, which is in the form of a liquid capable of application as drops and/or as a spray.

25. (canceled)

26. A product comprising an antimicrobial formulation according to claim 1.

27. (canceled)

28. A kit for preparing an antimicrobial formulation, the kit comprising sources of (a) a mono- or di-alkyl-substituted benzoquinone, a mono- or di-alkyl-substituted hydroquinone or a mixture thereof, wherein the alkyl group(s) are attached to carbon atom(s) of the cyclohexyl ring, and (b) a copper salt, together with instructions for combining the two compounds so as to make the formulation at or before the point of its intended application, and/or for the co-administration of the two compounds to a surface.

29-46. (canceled)

47. A method of treatment of a human or animal patient suffering from or at risk of suffering from a condition which is caused by, transmitted by and/or exacerbated by microbial, in particular bacterial, activity, the method comprising administering to the patient a therapeutically (which term includes prophylactically) effective amount of an antimicrobial formulation comprising (a) an alkyl-substituted benzoquinone, an alkyl-substituted hydroquinone or a mixture thereof, wherein the alkyl group(s) are attached to carbon atom(s) of the cyclohexyl ring, and (b) a copper salt.

48. A method according to claim 47, wherein the condition is acne.

49. A method according to claim 47, wherein the condition is a staphylococcal infection.

50. (canceled)

51. A method for controlling the growth of a micro-organism, the method comprising applying, to an area infected or suspected to be infected or capable of becoming infected with the micro-organism, a combination of (a) a mono- or di-alkyl-substituted benzoquinone, a mono- or di-alkyl-substituted hydroquinone or a mixture thereof, wherein the alkyl group(s) are attached to carbon atom(s) of the cyclohexyl ring, and (b) a copper salt, wherein the area to which the quinone and the copper salt are applied is a non-living surface.

52-55. (canceled)

56. A method for controlling the growth of a micro-organism, in a product which comprises or is suspected to comprise or is capable of comprising the microorganism, the method comprising incorporating into the product a combination of (a) a mono- or di-alkyl-substituted benzoquinone, a mono- or di-alkyl-substituted hydroquinone or a mixture thereof, wherein the alkyl group(s) are attached to carbon atom(s) of the cyclohexyl ring, and (b) a copper salt.

57-74. (canceled)

75. A method according to claim 47, wherein the condition is a skin or skin structure condition.

76. A method according to claim 47, wherein the condition is caused by, transmitted by and/or exacerbated by propionibacteria.
